(19) United States
(12) Patent Application Publication
(10) Pub. No.: US 2010/0268121 A1
(43) Pub. Date: Oct. 21, 2010

(54) Active support surface

(76) Inventor: John C. Kilborn, Winnetka, IL (US)

Correspondence Address: VARNUM, RIDDERING, SCHMIDT & HOWLETT LLP, 333 BRIDGE STREET, NW, P.O. BOX 352, GRAND RAPIDS, MI, 49501-0352, US

(21) Appl. No.: 12/661,485

(22) Filed: Mar. 18, 2010

Related U.S. Application Data
(60) Provisional application No. 61/161,195, filed on Mar. 18, 2009.

(51) Int. Cl. A61B 5/103; A61G 7/065; A61G 7/05

(52) U.S. Cl. 600/587; 5/652

(57) ABSTRACT

An active support assembly (30) includes a frame assembly (32) having subframes (34). Active devices (36) includes a flexible element (38) having a stationary end (40) attached to the frame assembly (42). An opposing end of each of the active devices (36) or flexible elements (38) also include a movable end (42). Electroactive polymer actuators (70) are organized into an array (68), with each actuator (70) aligned with a flexible element (38). Increases in the level of the activation signals cause the corresponding actuators (70) to expand, and forcibly extend the movable ends (42), with expansion of the flexible elements (38). The active support assemblies can be used with mattresses, overlays and seating for purposes of adjusting the same in response to the application of external forces caused by an occupant.

### CROSS-REFERENCE TO RELATED APPLICATIONS

This application claims priority and is based upon U.S. Provisional Patent Application Ser. No. 61/161,195, filed Mar. 18, 2009.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The invention relates to support assemblies for facilitating comfortable support of human individuals and, more particularly, to active support assemblies having means for movement in response to application of external forces.

2. Background Art

Subjects who are seated or recumbent for long periods of time are exposed to sustained mechanical forces. These forces can comprise a compressional and, in some cases, a vibrational component. The impact of these compressional and vibrational component forces on both subject comfort and soft tissue viability can be significant. Specifically, depending upon: (a) the design and construction of the device providing postural support to the subject; (b) the device's response to the load created by the subject; (c) the degree to which the device isolates the subject from external forces; and (d) the health of the subject, these sustained forces can lead to discomfort, poor circulation, muscle stiffness, inflammation, low back pain and/or pressure ulcers (PUs).

### Impact of Vibrational Forces

Subjects whose occupations expose them to sustained vibrational forces while seated are prone to low back pain. Such exposure is common among truck drivers and heavy equipment/industrial vehicle operators. These and similar occupations report a high prevalence of low back pain. Postural support devices often incorporate passive technologies, e.g., springs, to isolate subjects from external vibrational forces. Unfortunately, these solutions are effective only when the spring constant is matched to both the frequency of the vibrational force and the subject mass. If instead, the postural support device were to incorporate an active technology, capable of sensing and measuring the external vibrational force (frequency and amplitude), and producing an equal and opposing vibrational force to cancel it, its ability to isolate subjects from external vibrational forces would be independent of both vibrational frequency and subject mass.

### Impact of Compressional Forces

The body's natural response to compressive loading is to reposition itself. Subjects confined to seated or recumbent postures for long periods of time reposition themselves frequently to shift the weight born by their soft tissues, particularly those covering bony prominences, and relieve compressive loading. When this natural response is inhibited, as is the case with subjects who have impaired mobility and/or sensitivity, the soft tissues become subject to sustained compression.

Sustained compression of tissue triggers several pathophysiologic (i.e., disease progression) processes: localized ischemia (lack of blood supply in an organ or tissue); impaired interstitial fluid flow (impaired flow between cellular components) and lymphatic drainage; possible reperfusion injury; and sustained deformation of cells. In subjects who are confined to a bed or wheelchair for long periods of time, and whose tissue health is compromised by age, disease, injury, malnutrition, medical treatments or other factors, these pathophysiologic processes often lead to the development of PUs. Not surprisingly, a high prevalence of PUs has been observed in healthcare settings, where this cohort is concentrated. Aside from being painful, PUs can lead to: depression; loss of function and independence; increase in the incidence of infection and sepsis (poisoning caused by absorption of pathogenic microorganisms and their products into the blood stream); and ultimately require surgical intervention. As a consequence of all of the foregoing, various specialty mattresses and overlays have been developed for the healthcare market, where they are routinely prescribed for patients who are at high risk for developing a PU. In addition, various other devices have been developed for varying pressures associated with body support or other types of reduction of stress concentration.

### Specific Prior Art Regarding Specialty Mattresses and Overlays

For example, Rogers, U.S. Patent Application Publication No. 2008/0028532, published Feb. 7, 2008, is directed to improvements in mattresses or cushions which are enclosed in a functional membrane. The purpose of the invention is to control flexible, rigid or visco-elastic foam, springs, air, fluids, particulates, combinations thereof and foam density variations so as to meet varying force support needs. In addition, other concepts associated with support apparatus are disclosed which employ surface technology so as to modify basic support characteristics and an interface created therefrom with a supported body. The purpose is to support the body in an optimal manner.

More specifically, Rogers discloses various concepts associated with tissue trauma or tissue death. These concepts are shown in various drawings, which illustrate foam slabs 1 with a body 2 loading the foam 1 to a height 3. In FIG. 1 of Rogers, a part (c) illustrates a heavier body placed upon the same foam, which compresses to where equilibrium has been reached. In part (d), with a heavier load, a thickness 5 is shown which depicts the possibility of “bottoming-out.” Part (e) illustrates a bony body 6 placed upon a similar foam block 1. The resulting forces 7 are illustrated, where the bone 9 is penetrating the tissue and causing shear forces 8 up the side of the bone. This force can be shown to be directly related to the peak pressure 12, while the additional shear load at a tissue surface 11 and the surface tension 10 also contribute as shown in part (f). Specifically, part (f) depicts the breakdown of tissue at the surface 13, with the majority of damage being at the bone as illustrated at point 14.

FIG. 3 of Rogers illustrates a person's trunk section 1 placed on a support 27. Leg section 2 is also placed on the support 27, with the support consisting of corrugated cardboard for a low costing that can consist of two adjacent panels so as to allow rolling of the supported mattress or cushion. In the case of the mattress, “gatching” of the supported mattress is allowed, while providing the back with support, clearing the coccyx and ischial tuberosities of the patient, and allowing independent positioning of the legs. A foot pillow 3 is attached by a hinge 28 to the main cover of Dartex-like material used over the leg section. Material 4 and material 15 are outer edge materials, with cavities in between and below, so as to support the use of a collector 24 fitting within a sloping cavity. A rolled or folded transfer sheet 5 is attached to the cover 7 and the leg covering. The sheet can be folded back onto itself for the patient to be rolled upon and when in place, the sheet is pulled along with the patient for transfer to a wheelchair or the like. A sensor 6 is illustrated which can be built into the cover or can be a separate item. An outer fluid proof material 7 is vapor permeable and allows moisture to enter the inside foam if not protected. The material is RF welded on all edges, so as to assure fluid proof integrity. A self-inflating pillow 8 is attached by hinging the pillow to the cover material. The pillow 8 is filled with particular material so that it can be vacuum controlled, fluid inflated or time restrainted compressed so as to meet the patient's needs. Valving 10 is provided for purposes of self-inflating. Reference 11 illustrates the pillow in a deflated mode, and positioned over the end of the bedding. Element 12 illustrates a vent control system of the pillow. An inflated pillow is shown as element 13. Element 15 is an end filler of the cavity between the head and leg portion of the support system. Element 16 illustrates the back-sloping edge of the cut out running laterally across the mattress for pressure/shear relief of the Trochanter-coccyx-sacral area of the body fitting over the cavity 30.

Elements 17 are valves for controlling the positioning and functioning of a rotated foot piece 21 hinged at location 28 and used for purposes of supporting the patient's knees. Reference 18 illustrates the pillow-leg portion in the knee support position. Elements 19 are valves for purposes of controlling time constants, vacuum forming or inflating ability of various portions of the mattress. Reference 20 illustrates the same unit pushed up into place, and held by a hinge 28 and particulate material fluid vacated so as to form unit to feet, if needed. Reference 21 is a foot pillow positioned for normal foot control, as needed. Reference 22 indicates the valve positioning. Reference 23 illustrates a hinge point in the base unit, and element 24 illustrates a collector of waste material which can be removed from under the patient when appropriate. Reference 25 is a self-inflating pillow for positioning the collector 24. Reference 26 is a pneumatic sensor for purposes of measuring peak pressure/shear.

Reference 27 illustrates the composite baseboard. When a patient is in place on the mattress, the buttock portion of the body will be free of support due to the cavity 30 in which the collector 24 is placed. Reference 28 illustrates the hinged portion of the pillow 3. Reference 29 illustrates a zipper-like portion for placement of a stiffener, so as to ensure that the vacuum aspect pulls down under the patient, rather than allowing the vacuum to also raise the bottom of the cavity.

The mattress can be pneumatically controlled by valves at reference location 22 to any degree needed by the patient. The head can be elevated, rotated and controlled with the “softness” dictated by the inflation level of the mattress invention. The knees can be elevated through use of the rotating foot/leg unit. FIG. 4 illustrates the cavity function, with loading and vacuum control. FIG. 5 illustrates a pressure/shear transducer.

FIG. 6 illustrates three versions of the Rogers mattress concepts. These versions illustrate general use with mattresses, cushions, and for shipments of fragile goods. In brief summary, Rogers discloses the concept of what could be characterized as a pressure gradient dampening apparatus. The apparatus includes a foam body with foam force accommodation zones located at pre-selection positions. Dampening forces are applied to the foam body at or near the foam force accommodation zones. An enclosure member is also provided, which is impermeable to gas and fluid. Valve devices interface with the enclosure member for permitting introduction of an inflation medium into, or exhaustion of an inflation medium from, an interior space of the enclosure member. A pump is provided, which is interfaced with the valve devices, for facilitating introduction or exhaustion of the inflation medium.

Another example of a support system which may be utilized with mattresses is disclosed in Jones, U.S. Pat. No. 6,742,202 issued Jun. 1, 2004. The Jones patent discloses a support system with an array of telescoping columns, each being extended by a spring. The columns define a support plane for a body resting on the column array. The columns can move perpendicular to the support plane (as well as independently of each other), in response to the weight of a body resting upon the columns. Jones further discloses the concept that means for keeping the columns extended may be either passive or active. The columns may be moved independently by actuators connected to the columns. Also, the columns can be assembled at the modules, which, in turn, can be assembled into support systems of arbitrary size and shape.

More specifically, Jones discloses the use of telescoping columns 105 mounted to a substantially perpendicular common base 100 in a closely-spaced array. The telescoping column 105 includes an upper section 120 and lower section 110. The sections have appropriately and differently sized diameters, so that the sections can slidably engage one another. A spring 130 is positioned within the upper section 120, with the spring having an outside diameter equal to the outside diameter of the lower section 110. Jones discloses the concept that the spring 130 is used to provide the capability of extending the telescoping column 105. However, Jones also discloses that equivalents may be used, which Jones describes as: resilient substances such as rubber or plastic; balloons; hydraulic or pneumatic shock absorbers; or active means such as hydraulic, pneumatic or electric actuators. The base 100 formed of the array of telescoping columns 105 is mounted to a frame 300, so as to form a module 290. The array of telescoping columns 105 is described as providing the ability of the system to conform to relatively short-radius curves, concave or convex with respect to the array of columns 105. The columns 105 move independently of one another under the weight of a body resting upon the columns 105. Jones further discloses the concept of the telescoping rods being mounted in staggered rows, rather than congruent rows. However, this spacing requires one dimension of the finished array to be larger than the other, by a one-half column diameter. Jones further discloses the concept of the columns having a 0.95 inch diameter, with each column having an on-center spacing of 1.0 inches. Possible springs for use are disclosed as being stainless steel or zinc-plated, having a diameter of 0.77 inches with a length of 2.5 inches. A spring constant of 30 pounds per inch is also disclosed.

The assembled support system comprising the modules 290 forms a plane of support. The plane of support is defined by the extension of the telescoping columns 105. As a body rests upon the support system, the support plane is deformed in conformity with the shape of the body. Jones also discloses the concept that the modules may be assembled so as to form supports, mattresses or beds of predetermined sizes, simply by the use of the particular number of modules in different configurations.

Jones also discloses, as an alternative embodiment, the use of a coaxial actuator rod 240 connected to the upper section 120 and extending through the base 100. The rod 240 operates a linear position transducer 250, such as a servo motor, so as to produce a signal proportional to the displacement of the actuator rod. From this signal, velocity and acceleration of the actuator rod 240 can be calculated. The actuator rod 240 can be connected to an actuator, capable of forcing the actuator rod 240 (and the interconnected upper section 120) to move up or down in response to an externally applied signal.

Jones further discloses the concept of the telescoping columns 105 possibly being programmed so as to provide greater or lesser support at different parts of the plane of support. Also, the resistance in compression of the telescoping columns 105 could be programmed so as to follow a non-linear function, using negative feedback from the linear position transducers 250. In this regard, FIG. 10 illustrates an output signal from a linear position transducer 250 being received by a general purpose computer 430. The computer 430 can include a data bus which drives a servo amplifier 440 which, in turn, drives the actuator 260. The computer 430 could be programmed so as to command the actuators 260 to raise, lower or rotate a body relative to the support plane, for purposes of scanning operations.

Dimitriu, et al, U.S. Pat. No. 6,892,405, issued May 17, 2005 discloses a therapeutic mattress system and bed. The patent is relatively extensive, and discloses various types of features for providing rotational therapy, percussion therapy and pulsation therapy on a critical care bed frame with a low air loss patient support. The features of the mattress system are controlled with various types of feedback from particular sensors in the bed.

In one embodiment, Demitriu, et. al. disclose a medical bed with a head and foot section, and a longitudinal axis. A first inflatable enclosure is provided for laterally rotating the head section. This enclosure is described as an inflatable bladder for lifting one side of the head section relative to the opposite side. A second inflatable bladder is provided for laterally rotating the foot section in a second direction relative to the head section. The first and second inflatable bladders operate in two modes, with a first mode providing substantially no support to the mattress. The second mode provides support to the mattress, while imparting a rotating force.

Dimitriu, et. al. also disclose the use of a series of transversely-oriented inflatable cushions. Pressurized gas is in fluid communication with the cushions. A series of magnetic field strength sensors are responsive to changes in distance between upper and lower surfaces of the cushions, and a controller regulates the gas source in counter response to the distance detected by the magnetic field strength sensor. Also disclosed is the use of an electrically conductive baffle sheet which is positioned within the cushion interiors. Electrically conductive material is positioned approximate the interior lower surface of the cushions, and detection means are a responsive to the contact between the baffle sheet and the conductive material. A controller regulates the gas pressure in counter-response to detection of the contact. Dimitriu, et. al. further disclose the use of third inflatable enclosures positioned beneath a series of air sacs in a chest region of the mattress, for purposes of imparting a percussive force upward and through the air sacs during mattress rotation.

Guthrie, U.S. Pat. No. 6,745,996, issued Jun. 8, 2004 discloses an alternating pressure valve system which can be utilized for supplying fluid to alternating pressure air mattresses. In one embodiment, the valve system includes a blower with an air intake and an air outlet. A rotor valve assembly is connected to the air outlet from the blower. The valve assembly includes a housing with an air intake, air outlet and additional air outlet. A circular chamber is provided for receiving air from the air intake. A wedge-shaped rotor valve is rotatably contained within the circular chamber of the housing. The rotor valve can rotate so as to block the first air outlet, block the second air outlet or block neither of the outlets. Apparatus is provided for controlling the rotation of the valve, and the rotor valve is shaped such that the top of the valve completely blocks air flow to either of the first or second air outlets when the valve is positioned in front of the outlets, and the shaft of the rotor valve is recessed from the circular chamber so as to allow for some air to flow around the rotor valve into the air outlet that is being partially blocked by the rotor valve shaft. Various other concepts have also been developed within the prior art, relating to patient support. For example, Weinstenin, et al., U.S. Pat. No. 3,456,270 describes a patient support apparatus using air bladders. The supporting medium was described as water, and a lifting inflatable bladder interface was used for lifting the patient for transfer. Whitney, U.S. Pat. No. 3,802,004 would modify a patient immersion depth through what would be considered to be unique bladder arrangements inflated by air, without change of medium volume.

Hagopian, U.S. Pat. Nos. 5,072,468 and 5,068,935 describe bed frames using water as a base medium, with an air bladder on the upper surface to lower or raise the patient. The patents also describe the use of an inflated wedge for postural trunk control of the patient. In these foregoing patent references, approaches were utilized so as to attempt to reduce what could be characterized as “hammocking” over boney prominences that tend to negate the efficacy of the support medium. In this regard, it is noted that relatively modern water beds consist of water, a supporting envelope to “hammock” someone so that they do not sink into the bed, and appropriate baffling or channeling for stability of the water.

Air support has been used for a substantial period of time, in which air has been compressed, blown or applied for patient support. Hart, U.S. Pat. No. 1,772,310 is an early disclosure of a technique for altering the fluidic support points on the body, by controlling the time each support point is to be activated. This occurs while still limiting interface pressure to an acceptable value. In the same patent, Hart also introduced a method using the air for purposes of patient turning. In Whitney, U.S. Pat. No. 3,148,391, a support method utilized air control and also introduced temperature control of the interface.

Ford, U.S. Pat. No. 4,711,275 was one of the earlier patents to disclose the concept of inflating and deflating arrays of air cells through independent air compressors. In this manner, an alternating pressure support system was achieved. In Krouskop, U.S. Pat. No. 4,989,283, the height of supporting bladders were controlled by measuring changes in cell configuration through the use of a microprocessor. The microprocessor used input from internal bladder sensors so as to control appropriate valving to pressure sources and exhausts so as to maintain each bladder at a particular referenced height.

Carrier, Canadian Patent No. 1,035,000 disclosed a surgical table where individual bladders of air are positioned so as to keep the bony prominences clear of the table. Each bladder was independently inflated to the desired pressure, and then covered by a “forgiving” cover.

Armstrong, U.S. Pat. No. 2,998,817 appears to be one of the earlier patents which disclose an inflatable massaging and “cooling” system. As materials were developed which had leak rates suitable for beds, the current approach of using low air loss mattresses evolved, with these mattresses using what are characterized as vapor-permeable materials. Such materials typically consist of nylon, backed with a material such as a film of urethane or vinyl.

Hess, U.S. Pat. No. 4,638,519, disclosed the use of shaped bladders using materials with appropriate individual bladder control and methods of bladder attachments with air supplies. Goode, U.S. Pat. No. 4,797,962 used the process of controlling these types of air bladders in groups as a means of modifying support pressure under portions of the body. Wilkinson, U.S. Pat. No. 5,070,560 disclosed the concept of body support through the use of foam on top of slats placed on the top of air cylinders.

Reswick, U.S. Pat. No. 3,803,647, disclosed the use of a fluid comprising a mixture of barium sulfate ore and water for the support medium, with a loose fitting lifting interface sheet as the top member of the unit. The solution of barites consists of a relatively high specific gravity and, accordingly, support the body without immersion problems. Thompson, U.S. Pat. No. 4,357,722, disclosed the use of a flexible, open mesh within a special bed frame to support a patient interfacing medium, so as to change tension of support under various portions of the body.

Hargest, et al, U.S. Pat. Nos. 3,428,973 and 3,866,606, disclosed the use of fluidized beads to create a high specific gravity. The beads consist of micro-balloons and are fluidized by an air plenum chamber placed at the base of the beads, and separated by appropriate filtering. Fluidization depends on the pressure drop across the supporting beads and that of the filtering system.

Lacoste, U.S. Pat. No. 4,481,686, also disclosed the use of beads, and includes concepts associated with bacteria control through bead selection. The use of beads for purposes of support is also discussed in Goodwin, U.S. Pat. Nos. 4,564,965; 4,672,699; and 4,776,050.

The use of beads is also referenced in Viard, U.S. Pat. No. 5,402,542. The Viard patent discloses the use of a programmable EPROM and heat exchanger to control bead system component temperatures.

As a substitute for beads, it is also known to employ river sand. In addition, combined uses of gels and air have been utilized in place of the fluidic bead systems and relatively thin beds. However, because of the nature of most gels, the accommodation of the gels to relatively high forces is somewhat limited.

All specialty mattresses and overlays developed to this point operate on the same basic principle: by redistributing patient weight over a larger surface area, they moderate the tissue interface pressures that ultimately lead to PU development. In general, these mattresses achieve pressure redistribution by means of a compliant support surface constructed of air- or fluid-filled cells. Two types of specialty mattresses extend this concept further. One type of specialty mattress is typically referred to as a “low air loss mattress.” Low air loss mattresses have a perforated surface that allows air to slowly escape and wick moisture away from patient skin. A second type of specialty mattress is commonly referred to as an “alternating pressure mattress. Alternating pressure mattresses adjust pressures across the tissue interface by continuously inflating and deflating alternate rows of cells, where cycle times are generally on the order of minutes.

An example of an alternating pressure air mattress is shown in FIG. 1 as embodied within an alternating pressure mattress system 10. The known alternating pressure mattress system 10 can include a conventional and stationary base 12. A specialized alternating pressure mattress 14 is positioned on top of the base 12. The pressure mattress 14 can have a head portion 16 which may take various configurations for supporting the patient's head. The pressure mattress 14 also includes a cell portion 18. The cell portion 18 includes a series of individual inflatable/deflatable cells 21 which can take on a number of various types of configurations. However, the cells 21 must have an elastic bladder or similar configuration which permits inflation and deflation with a fluid, including air and other gaseous fluids. The cells 21 are arranged in cell rows 20. Individual cell rows 20 are identified in FIG. 1 as cell rows 20a, 20b, 20c, 20d and ongoing, including cell row 20g et al. An air vacuum/pump 22 of a conventional nature can be attached through an air hose 24 to the pressure mattress 14. Specifically, the air hose 24 can be connected to a series of valves (not shown) which can be controlled through various means so as to continuously inflate and deflate the cells 21 of alternate cell rows 20. The valves (not shown) can be controlled through various means, including pneumatic, electronic or programmable means. As earlier stated herein, typical cycle times for continuously inflating and deflating cells 21 of cell rows 20 on an alternate basis are generally on the order of minutes.

As apparent from the prior discussion and the prior art references cited therein, existing specialty mattresses have several drawbacks. They are sometimes perceived as noisy, uncomfortable and unreliable. All classes of air- and fluid-filled mattresses are contraindicated for patients with unstabilized spinal injuries. Such patients consist of one of the groups which is most susceptible to PU development. This is as a result of the mattresses' inherent lack of stability. This lack of stability also complicates patient transfers and CPR administration, and hinders a patient's efforts to self-mobilize. Furthermore, specialty mattresses are expensive, ranging from $800 to $6000 per system, depending on the feature set.

Perhaps the greatest shortcoming of specialty mattresses, however, is that by focusing exclusively on tissue interface pressures, they ignore the principal causal factor associated with the most severe PUs. That factor is sustained compression of deep muscle layers covering bony prominences. This type of compression seems to occur even when interface pressures are moderate and manageable by the surface tissues. It has been well established that tissue damage is often apparent following prolonged loading, even at relatively low level pressure intensities. An external (tissue interface) pressure of 50 mm Hg may rise to over 200 mm Hg at a bony prominence, leading, with time, to deep tissue destruction, which may not be evident on the surface of the skin. Regular relief from high pressures in the at-risk patient, then, is essential to prevent pressure ulceration. Alternating pressure mattresses are likely the most effective of the specialty mattresses in relieving compression of the deep tissues as they not only (statically) redistribute tissue interface pressures, but actively modulate them. However, because the cells that compose alternating pressure mattresses are large, the relief they provide to the deep tissues is imprecise. Also, inherent in the location and orientation of these cells are assumptions about where PUs are most likely to develop, e.g., the sacrum, the heel, etc., assumptions that may be valid when a patient is supine and positioned precisely on the mattress, but not when they shift locations or lie prone or on their side. Moreover, there is a risk, again due to cell size, that when one row of cells is deflated, localized pressures over an adjacent (inflated) row of cells will attain unsafe levels, as that portion of the body is asked to bear more of the patient's total body weight. The performance of an alternating pressure mattress then depends largely on how well its active cell rows are aligned with patient bony prominences at any point in time. Considering that at-risk patients are supposed to be turned by staff at least every 2 hours, the degree of alignment is not assured. Finally, and perhaps most importantly, the cycle times of alternating pressure mattresses are long (measured in minutes) and thus may significantly prolong tissue recovery times in high risk patients.

### Pressure Ulcer Etiology

Through research combining animal studies with sophisticated computer modeling, experts have lately begun to understand the complexity of PU causation. There is strong evidence that surface (interface) pressures are not representative of the internal mechanical conditions inside the tissue, which are most relevant for tissue breakdown, especially when tissue geometry and composition are complex and surface pressures result in highly inhomogeneous internal mechanical conditions. This is the case adjacent to bony prominences. It is now accepted that deep tissue PUs (the most severe kind of PUs) arise in deep muscle layers covering bony prominences and are mainly caused by sustained compression of the tissue. Studies have also shown that muscle tissue is more susceptible to mechanical loading than skin and that tissue recovery from compressive loading of several minutes duration is much slower among elderly patients (e.g. greater than 2 minutes) than other patient groups. Moreover, soft tissues exhibit viscoelastic behavior and thus the nature of the recovery will depend on the rate and time of loading as well as its magnitude. Short term loading generally produces elastic recovery, while long term loading results in the creep phenomenon and requires a longer time for complete tissue recovery.

Taken together, these developments suggest a solution that eliminates the sustained compression of muscle tissue that leads to lengthy tissue recovery times and tissue damage. Such a solution would need to: (a) modulate not only tissue interface pressures, but also pressure vectors in deep tissues; and (b) affect this modulation with greater precision, responsiveness and frequency than do existing specialty mattresses to minimize loading duration and promote elastic tissue recovery. Comfort

Seating products that incorporate a massaging capability have been available for several years. Massage chairs for the home and high-end automobile seating are salient examples. Most of these products use electromechanical actuators to generate the massaging motion, which limits the precision, speed, flexibility and dexterity of these motions, as well as the ability of the user to control them. Also, because electromechanical actuators are bulky, the technology does not lend itself to broader application. A less bulky solution that makes improvements in precision, speed, flexibility, dexterity and user control would provide a higher level of performance, increase user satisfaction and enable broader application.

Several mattresses purport to offer superior comfort to the occupant, including standard coil mattresses, foam mattresses, viscoelastic foam mattresses, air mattresses and water mattresses. Each has advantages and disadvantages as a sleep surface. In general, high end mattresses attempt to strike an optimum balance between stability, firmness and peak pressure reduction. But mattresses are often shared by occupants with widely varying comfort preferences. Further, an occupant's comfort preferences may evolve over time, so the likelihood that a particular type of mattress will satisfy its occupants for the duration of its warranty period (typically 10 years) is low. Some existing mattresses incorporate two separate banks of air-filled cells, wherein the firmness of each side can be adjusted independently by changing the pressure of the corresponding bank. However, the cell size in these mattresses is large and, consequently, the adjustments are gross in nature.

There is also an opportunity to improve the postural support provided by a sleep surface by adapting its contours in response to changes in occupant positions. For example, a slight convexity centered under the thoracic back can improve respiration in supine occupants. Also, a convexity in the popliteal region can reduce strain on the lower back in supine occupants. Existing mattresses are incapable of determining occupant positions and unable to adjust their contours.

A technology that enables a mattress to mimic different sleep surfaces and create multiple surface zones, such that each mattress occupant may individually select a preferred sleep surface at the flip of a switch, would increase occupant satisfaction. A technology that detects occupant positions and adapts mattress contours in response would provide relatively optimum postural support.

### Concept for a Solution

One concept for a solution to the problems described in the foregoing paragraphs is a densely populated array of small active devices, wherein these active devices are simultaneously capable of motion and sensing, and wherein the motion of each device is controlled independently by a microcontroller in response to external forces sensed by the device. Such a concept exploits the same paradigm that has led to innovations in several other applications, including radar (where an active phased array is used to ‘steer’ an antenna with no moving parts) and ultrasound (where a phased array of piezoelectric crystals is used to emit ultrasound signals and sense their reflections). Device designs based on arrays of elements are inherently flexible, scalable and cost-effective. Moreover, their functional and performance attributes are largely embodied in software algorithms and thus can be extended dramatically via software upgrades.

Such a concept, however, demands an enabling technology with a unique set of attributes. To isolate vibrational forces, the array must be able to sense high frequency vibrational forces and generate device movements of equally high frequency. To moderate compressional forces, the array must provide precise relief that is independent of subject position, implying an arrangement of hundreds of small motive and sensing devices. To mimic various sleep surfaces, the array must continuously sense changes in subject loads and rapidly generate complex device movements in response. It follows then that this conceptual array must comprise individual devices that are not only small, but low cost and lightweight, and that operate with speed and precision. Because the array will be active when a subject is sleeping, the devices must also operate silently.

A cursory examination of the technologies used in conventional specialty mattresses and overlays reveals their limitations as enabling technologies for these new concepts. As previously described herein, a number of these technologies exist in various prior art references.

### Air- or Fluid-Filled Cells

An implementation based on air- or fluid-filled cells would require hundreds of cells and valves, and a complex and expensive routing system (for air or fluid) to control cell movements independently. Achieving the combined force and speed necessary for effective relief, given the size constraint imposed on individual cells, would be very difficult. Also, aside from presenting a daunting manufacturing challenge, such an implementation would be noisy.

### Electromagnetic Actuators

Electromagnetic actuators are generally large, heavy, expensive, noisy and power-hungry. While they can generate considerable linear force, they do not provide the combination of speed and precision necessary for comfortable, effective relief.

### Pneumatic Actuators

An implementation based on pneumatic actuators would require hundreds of valves and a complex and expensive routing system (for the compressed air that drives these actuators) to control actuator movements independently. Aside from being noisy, and similar to electromagnetic actuators, pneumatic actuators do not provide the combination of speed and precision necessary for comfortable, effective relief.

### A New Enabling Technology: Active Materials

Several classes of active materials, including shape memory alloys, ferromagnetic shape memory alloys, magnetorheological fluids, magnetorheological elastomers, electrorheological fluids, electrorheological elastomers, electroactive polymers, and piezoelectric materials, offer advantages over the conventional technologies used in existing specialty mattress and overlay designs. Specifically, these active materials have the following attributes:

1. High energy density.
2. Excellent bandwidth.

### 3. Simplicity.

### 4. Scalability.

5. Silent operation.

### 6. Precision.

7. Light weight (in some cases).
8. Low cost (in some cases).

One of these classes of active materials, namely electroactive polymers, appears to be of particular interest in achieving the solutions desired in accordance with the invention of the present application.

### Electroactive Polymers

Electroactive polymers (EAPs), also known as artificial muscles, are a class of materials that deform significantly under the application of an electric potential and resume their original form when this potential is removed. EAPs provide strains (displacement per unit length) and forces (actuation pressure per density) on par with human muscle tissue. There are two types of EAPs, namely ionic EAPs and electronic EAPs. Ionic EAPs work on the basis of electro-chemistry (the mobility or diffusion of charged ions) and are active at very low voltages. They need to be kept wet and so must be sealed with a protective coating. Electronic EAPs are activated by high electric fields (on the order of 1 to 5 kilovolts with correspondingly low currents). Advantages associated with electronic EAPs include the following: relatively quick reaction; delivery of strong mechanical forces; the absence of a requirement of a protective coating; and almost no current is required to hold a position.

EAP technology offers several advantages over the conventional technologies used in known specialty mattress and overlay designs. These advantages include the fact that EAPs are relatively inexpensive, are relatively silent in operation, and are lightweight. In addition, EAPs consume very little power, and enable the construction of relatively small devices. EAPs are also capable of relatively precise (on the order of 1 micron) and rapid (greater than 300 Hz) movements. Still further, EAPs can be utilized not only as motive devices, but also as sensors.

### SUMMARY OF THE INVENTION

In accordance with the invention, an active support assembly is adapted for use with mattresses, overlays and seating. The support assembly includes a frame assembly and a series of support plates. An array of active devices is also included. The active devices comprise stationary ends fixedly attached to the frame assembly, as well as movable ends operably attached to the support plates.

The active devices further comprise electroactive polymer actuators. A first section of each electroactive polymer actuator of a subset of the actuators is attached to the stationary end of the active device. A second section of each of the electroactive polymer actuators of the subset of actuators is attached to the movable end of the active device. Each of the active devices functions as a sensor. Movement of the movable end of each of the active devices resulting from the application of an external force to the support plate attached to the active device effects a change in an attribute of the actuator, wherein the change in the attribute is measurable. Still further, each of the active devices also functions as an actuator. Application of an activation signal to the actuator effects a change in a further attribute of the actuator. The change in the further attribute forcibly moves the movable end of the active device in the attached support plate.

The active support assembly also includes a controller in operative communication with each of the actuators of each of the active devices. The controller continuously measures the change in the attribute of the actuator, due to movement of the movable end of the active device and the attached support plate. Further, the controller uses the measured change in the attribute so as to compute a change in acceleration, velocity and position of the movable end, and in the magnitude of the applied force. The controller uses the change in acceleration, velocity and position of the movable end and the magnitude of the applied force to selectively apply an activation signal to the actuator, and effect a change in the attribute of the actuator. The change in the attribute results in a desired change in the acceleration, velocity and position of the movable end of the active device and the attached support plate, and in the magnitude of the resistive force. Further, the support plates form a plane of support under the application of certain of the activation signals and certain of the external forces.

The frame assembly includes a rigid or semi-rigid layer. The assembly also includes a compliant layer. The compliant layer is configured to comply with larger, slower forces applied externally and resist smaller, faster forces generated internally by the active devices under application of the activation signals. The external forces are mechanically coupled to the frame assembly through linkage of the support plates to the active devices, and to the frame assembly.

The array of active devices can be configured as a series of sub-array layers. Each of the layers includes a subframe fixedly attached to the frame assembly. The layers also include a series of extension rods having first and second ends. The active devices of each sub-array layer are offset at least one radii from the active devices of all succeeding sub-array layers.

The active devices have stationary ends fixedly attached to the subframe, and movable ends operably attached to the first ends of the extension rods. The extension rods penetrate all succeeding sub-array layers through intersects of the sub-array layer active devices. The second ends of the extension rods are operably attached to the support plates. The lengths of the extension rods are configured so as to dispose of the support plates on a plane under application of certain activation signals and certain external forces. Each plane is combined with planes of succeeding sub-array layers, so as to form a single plane of support.

The frame assembly comprises a series of modules. Each of the modules is characterized as a modular frame assembly and is rigidly or pivotably attached to others of the modular frame assemblies, so as to form a multi-frame assembly. A compliant material is fixedly inserted between adjacent ones of the modular assemblies which are attached to each other. The compliant material compresses when faces of the modular frame assemblies are parallel, and expands so as to fill space created when the faces of the modular frame assemblies are not parallel. The expanded material forms a continuous support surface between adjacent ones of the attached modular frame assemblies.

The support plates can be pivotably mounted to the movable ends of the active devices. The support plates can pivot in response to application of a non-uniform external force. The support plates can also be pre-loaded, so as to assume positions orthogonal to lengthwise directions of active devices to which the support plates are mounted, in the absence of external forces. The pre-load of the support plates consists of one or a combination of at least two of the following means for pre-loading: spring; air-filled cell; gel-filled cell; fluid-filled cell; foam cell. Further, each of the support plates can include a layer of compliant material, with the material covering a layer of rigid or semi-rigid material. The support plates can also be joined together by compliant connector ribbon, so as to form a network of support plates. The support plates and the connector ribbon can comprise a single sheet of the same material. The material thickness of the single sheet can be configured so as to effect different levels of compliance for the support plates and the connector ribbon. Still further, the size and shape of perforations in the single sheet of the same material can be configured so as to effect different levels of compliance for the support plates and the connector ribbon.

Each of the active devices includes a resistor in series with a corresponding one of the actuators. Further, each of the actuators can form a flexible capacitor. In this manner, each of the resistors and each of the actuators forms a resistor-capacitor circuit. Further, each of the resistors can be configured so as to establish a time constant of the corresponding resistor-capacitor circuit.

In accordance with further aspects of the invention, each of the active devices can comprise at least one sensor so as to measure the change in the attribute of the corresponding actuator. Each of the sensors can consist of one of the following elements, or a combination of at least two of the following elements: capacitance sensor; current sensor; voltage sensor; position sensor; accelerometer.

Still further, the support assembly can comprise a stacking or bonding of multiple layers of the actuators, so as to provide for a set of multi-layer actuators. As a result of the stacking or bonding, the actuators are capable of exerting relatively greater forces than a single layer actuator under the application of an activation signal.

Still further, each of the active devices can include a flexible element. The flexible element can be an energy storage and return element, having a movable end and a stationary end fixedly attached to the frame assembly. Each device can also include an output cap and a mask comprising a section of material having an opening so as to receive the flexible element. The actuators lay between the flexible element and the mask. The mask is positioned in a manner so that it is lowered over the flexible element and bonded to the frame assembly. Accordingly, the actuator is stretched over the flexible element. The output cap is operably attached to the movable end of the flexible element. In this manner, a non-active portion of the actuator lies between the movable end of the flexible element and the output cap. The actuator is also configured so as to forceably retract the movable end and the output cap. This compresses the flexible element. Alternatively, the actuator can forcibly extend the movable end and the output cap, thus expanding the flexible element under application of selective activation signals. The output cap is operably attached to the support plate.

Still further, each of the flexible elements can consist of one or more of the following elements: spring; air-filled cell; gel-filled cell; fluid-filled cell; foam cell. Still further, each of the active devices can be configured as an active device section, with each of the sections including a flexible element section, a mask section, an actuator section and a series of output caps. The flexible element section can comprise an array of flexible elements. Each of the flexible elements has a stationary end fixedly attached to the frame assembly and the movable ends. The mask section can comprise a single section of material having a series of openings disposed so as to receive the flexible elements in the flexible elements section. The actuator section comprises a single section of material having a plurality of the actuators. The actuators are aligned with the flexible elements in the flexible elements section. Each of the actuator sections is configured so as to lie between the flexible element section and the mask section.

The mask section is lowered over the flexible element section, and bonded to the frame assembly. This causes the actuators to be stretched over the flexible elements in the flexible element section. The output caps are operably attached to the movable ends of the flexible elements. In this manner, a non-active portion of each of the actuator sections lies between the movable ends of the flexible elements and the output caps.

The actuators are configured so as to forcibly retract the movable ends and the output caps, thereby compressing the flexible elements or, alternatively, the actuators forcibly extend the movable ends and the output caps, thus expanding the flexible elements under application of selected activation signals.

Each of the active devices can be characterized as comprising an electroactive polymer push-pull actuator, with each of the push-pull actuators comprising an output shaft, output disk and first and second outer frames. Also, each push-pull actuator comprises first and second electroactive polymer actuators. The first polymer actuator is suspended between the first outer frame and the output disk. The second polymer actuator is suspended between the second outer frame and the output disk. The second outer frame is parallel to the first outer frame and offset from the first outer frame by a spacer. The first outer frame and/or the second outer frame are configured as the stationary end of the active device, with the stationary end being fixedly attached to the frame assembly.

The first polymer actuator exerts a pulling force on the output disk in one direction or, alternatively, the actuator exerts a pushing force on the output disk in an opposite direction under an application of selected activation signals. The second polymer actuator exerts a pulling force on the output disk in one direction or, alternatively, exerts a pushing force on the output disk in an opposite direction under application of selected activation signals. Directions and magnitudes of the forces exerted by the polymer actuators on the output disk are configured so as to forcefully move the disk. The output shaft includes a first end operably attached to the disk, and a second end configured as a movable end of the active device. The movable end is operably attached to the support plate.

Each of the push-pull actuators can be biased with a flexible element. The flexible element can be an energy storage and return element, and is optionally compressed. The flexible element includes a stationary end connected to one or more of the following elements: frame assembly; subframe; first outer frame; second outer frame. The flexible element also includes a movable end operably attached to the output disk of the push-pull actuator.

Each of the active devices can include a stack of at least two polymer push-pull actuators. Each of the additional actuators can be configured so as to increase displacement produced by the active device. Further, each of the active devices comprises an electroactive polymer roll actuator, each of the roll actuators comprising two electroactive polymer actuators, a mounting cap, an output cap and a flexible element. The flexible element can include an energy storage and return element, with the flexible element being in a compressed or non-compressed state. The two polymer actuators are wrapped around the flexible element so as to form a cylinder having first and second ends. The first end is configured as the stationary end of the active device, and the second end is configured as the movable end of the active device. The mounting cap is attached to the stationary end and fixedly attached to the frame assembly. The output cap is attached to the movable end and operably attached to the support plate. The two polymer actuators are configured so as to forcibly retract the movable end and the output cap, thus compressing the flexible element or, alternatively, the two polymer actuators forcibly extend the movable end and the output cap, thus expanding the flexible element, under application of selected activation signals.

The controller includes a series of controller means for selectively applying activation signals to the active devices in response to changes in the accelerations, velocities and positions of the movable ends and in magnitudes of the external forces. The controller also includes means operable by a user with the capability of selecting ones of the controller means to be used for selectively applying the activation signals.

The controller means can include one or more of the following means: means for sensing and relieving sustained compression of tissues occurring in occupants of the mattresses, overlays and/or seating; means for mimicking a response of a passive support surface to the externally applied forces; means for generating surface vibration so as to reduce a coefficient of friction of the support surface; means for sensing and reducing external vibrational forces communicated to the occupant; means for determining a location of the occupant on the support surface; means for identifying a probable posture of the occupant and recording changes in the probable posture within a patient electronic medical record; means for generating an alert in the event that the probable occupant posture does not change for a predetermined interval of time; means for facilitating user-directed massage; means for adopting support surface contours in response to changes in the occupant location and the probable occupant posture; means for controlling firmness and stability of the support surface; and means for synthesizing one or more responses by the support surface to the externally applied forces.

In accordance with another aspect of the invention, the support surface is divided into a series of zones. Each of the zones may be acted upon by differing ones of the controller means in a simultaneous manner.

In accordance with a further aspect of the invention, the invention includes a method for sensing and relieving sustained compression of tissues occurring in occupants of mattresses, overlays and/or seating. The method uses an array of active devices having stationary ends fixedly attached to a frame, and movable ends configured so as to form a support surface. The method includes detecting movements of the movable ends and outputting sensor signals associated with the detected movements. The detection is based on deformation of electroactive polymer actuators. The method further includes reading the sensor signals and computing magnitudes of external forces associated with movements of the movable ends. Also, locations of peak forces are identified, based upon the readings of the sensor signals.

Activation signals are selectively applied to the active devices. The activation signals cause the active devices to forcibly move their movable ends. The movements are generated by deformation of the actuators.

The active devices are caused at and proximal to the locations of the peak forces to retract their movable ends by variable amounts. This reduces resistive forces on occupant surface tissues at and proximal to locations of peak forces. Further, the method includes optionally causing the active devices distal to the locations of the peak forces to extend their movable ends by variable amounts. This increases the resistive forces on the occupant surface tissues at locations distal to the peak forces. Further, velocities of the movable ends are configured so as to limit impact forces on the occupant, until the locations of the peak forces change or a predetermined set time interval has elapsed. The time interval is capable of being set to ranges of seconds to minutes. The method further includes selectively updating the activation signals, so that the updated signals cause the active devices having movable ends in a retracted state to extend the movable ends by variable amounts, so as to increase the resistive forces on the occupant surface tissues at corresponding locations. Further, velocities of the movable ends are configured so as to limit the impact forces on the occupant, wherein the updated activation signals optionally cause the active devices having movable ends in an extended state to retract by variable amounts, so as to reduce the resistive forces on the occupant surface tissues at corresponding locations. Still further, the method includes effecting changes in magnitudes and directions of stress vectors in occupant deep tissues. This occurs through changes in the resistive forces on the occupant surface tissues and interrupting sustained compression of the deep tissues.

Applicant's invention also includes a method of mimicking of a passive support surface to externally applied forces, using an array of active devices having stationary ends fixedly attached to a frame, as well as movable ends configured so as to form a support surface. The method includes detecting movements of the movable ends and outputting sensor signals associated with the detective movements. The detection is based on deformation of electroactive polymer actuators. The method further includes reading the sensor signals and computing magnitudes of external forces associated with the movements of the movable ends. Activation signals are selectively applied to the active devices, and the signals cause active devices to forcibly move the movable ends by variable amounts. The movement is generated by deformation of the polymer actuators. Still further, the method includes computing direction and velocity of the forcible movement of each of the active devices using principles of superposition, as applied to an aggregate of direct and indirect impulse responses of the active device. The direct impulse response is defined by a position-versus-time curve of the movable end of the active device in response to an external force applied to the active device. The indirect impulse response is defined by a position-versus-time curve of the movable end of the active device in response to an external force applied to an adjacent one of the active devices.

The direct impulse response of the active device is based on a measured response of the passive support surface to a high amplitude, short duration force applied at a location coincident with a location of the active device in the array of active devices. The indirect impulse responses of the active device are based on measured responses of the passive support surface to a series of high amplitude, short duration forces sequentially applied at locations coincident with locations adjacent active devices in the array of active devices. The high amplitude, short duration forces approximate an impulse function.

A further method in accordance with the invention includes adjusting the firmness and stability of the mattresses, overlays and/or seating. The method also includes selecting a desired level of firmness and stability, and decreasing amplitude of the direct impulse responses in response to an increase in firmness. Amplitude of the direct impulse responses is increased in response to a decrease in firmness. Decreasing the amplitude and/or length and/or oscillations of the indirect impulse responses in response to an increase in stability is also accomplished. In addition, the method includes increasing the amplitude and/or length and/or oscillations of the indirect impulse responses in response to a decrease in stability.

The method can further include synthesizing the responses of the mattresses, overlays and/or seating to the externally applied forces. In addition, the method can include defining amplitudes, frequencies and damping of oscillations of the direct impulse responses, and defining amplitudes, frequencies and damping of oscillations of the indirect impulse responses.

Still further, the method can include sensing and reducing external vibrational forces communicated to occupants of seating, using an array of active devices having stationary ends fixedly attached to a frame, and movable ends configured so as to form a support surface. The method includes detecting movements of the movable ends, and outputting sensor signals associated with the detected movements. The detection is based on deformation of electroactive polymer actuators. Still further, the method includes reading the sensor signals and identifying components of the detected movements that are periodic on the basis of the sensor signals, and also measuring periods, amplitudes and phases of the periodic movements of the movable ends. Oppositional movements are computed, where each of the oppositional movements is configured so as to have the same period and amplitude as one of the measured periodic movements, and a phase difference of 180° with the periodic movement.

Activation signals can be selectively applied to the active devices, and cause the active devices to forcibly move their movable ends. The movement can be generated by deformation of the polymer actuators. Further, principles of superposition can be used to compute the period, amplitude and phase of the forcible movement of each of the active devices, where the principles of superposition are applied to an aggregate of the oppositional movements computed for the active device.

Methods in accordance with the invention also include generating surface vibration in mattresses, overlays and/or seating so as to reduce a coefficient of friction of a support surface, using an array of active devices having the stationary ends and movable ends. Activation signals are applied to the active devices to forcibly move the movable ends, the movement being generated by deformation of the polymer actuators. The movable ends are extended and retracted at frequencies of at least 10 Hz. For specific intervals of time, movable ends of one group of the active devices are retracted, and movable ends of a second group of the active devices are extended. An area of surface contact is reduced between an occupant and the mattresses, overlays and/or seating.

The method for determining the location of occupants of mattresses can include reading the sensor signals and, based on the sensor signals, computing pressure distribution across the support surface and computing the location of the occupant based on the pressure distribution. Also, the method can include processes for facilitating massage, including the step of selecting a massage tool icon from a series of icons presented on a pressure-sensitive graphical display. The massage tool icon can be dragged across an image of a body presented on the graphical display. A location can be selected for massage and adjusting forces are applied to the graphical display so as to select an intensity for the massage. The computed location of the occupant can be used to identify the active devices corresponding to the selected location. Activation signals can be selectively applied to the corresponding active devices, where the activation signal levels are based on selected massage intensity. Through the activation signals, the active devices can be caused to forcibly move their movable ends, with the movement being generated by deformation of the actuators.

Methods in accordance with the invention also include processes for identifying a probable posture of the occupant. Pressure distribution is computed, and correlated against a series of pre-load pressure distributions. The pre-loaded pressure distributions correspond to a series of unique occupant postures. A probable occupant posture is selected based on values of correlation coefficients determined from the correlation of the computed pressure distribution. The change in the probable occupancy posture can be recorded in an electronic medical record.

Methods in accordance with the invention can also include generating an alert in the event the probable occupancy posture does not change for a predetermined time interval. The method can also include processes for adapting contours of the support surface based on changes in the occupant location and the probable posture. For certain occupant locations and posture, the active devices can be retracted or extended at the certain locations.

Certain ones of the active devices can be extended which are located in a thoracic back region of a supine occupant, so as to improve respiration of the occupant. Certain ones of the active devices can be extended which are located in a popliteal region of a supine occupant, so as to reduce lower back strain.

Certain ones of the active devices can also be extended, which are located forward of ischial tuberosites of a reclining occupant, so as to prevent sliding. Devices can also be extended which are located lateral to a thorax of a seated occupant, so as to provide lateral support.

The Applicant's invention can also include a system for relieving sustained compression of tissues occurring in occupants of mattresses, overlays and/or seating. The system includes a frame and a series of active material base devices having stationary ends fixedly attached to the frame and movable ends forming a support surface. Movement of any one of the movable ends due to the application of an external force effects a change in an attribute of an active material. The change is measurable. Means are also provided for effecting a change in the active material attribute through application of an activation signal to the material. The change in the attribute forcibly moves the movable end. Controller means are in an operative communication with the active material, and continuously measured changes in the attributes due to movement of the movable ends. The controller means use measure changes in the attributes so as to compute changes in positions, velocities and accelerations of movable ends, and magnitudes of external forces. The changes are utilized to selectively apply activation signals to the active material and effect changes in the attributes of the material. The changes in the attributes cause desired changes in the accelerations, magnitudes and positions of the movable ends, and in the magnitudes of resistive forces.

In accordance with another aspect of the invention, controller means are in operative communication with sensors and actuators, and continuously sample information provided by the sensors so as to compute changes in positions, velocities and accelerations of movable ends, and in magnitudes of externally applied forces. Further, changes in attributes of the active material can result in changes in the shape and behavior of the support surface. Measurements of changes in the attribute can be provided to the controller means to selectively apply activation signals to the material. In accordance with another aspect of the invention, the means for controlling changes in the positions can control such changes within one micron. Also, changes in the positions can occur at velocities of up to at least 12 meters per second. Still further, changes in the velocities can occur at accelerations of up to 7200 meters per second². Also, audible sound produced by the changes in the position and the velocities can be less than 1 db.

### BRIEF DESCRIPTION OF THE DRAWINGS

The invention will now be described with respect to the drawings in which:

FIG. 1 is a perspective view of a prior art mattress system utilizing principles of alternating pressure;

FIG. 2 is a diagrammatic view illustrating four subframe layers of materials which may be utilized with an active support surface in accordance with the invention;

FIG. 3 is a diagrammatic cross section illustrating one embodiment of a sensing assembly utilizing Hall effect sensor principles;

FIG. 4 illustrates one embodiment of a permanent magnet which may be utilized with the active support surfaces in accordance with the invention;

FIG. 5 illustrates an embodiment of a linear Hall Effect switch which may be utilized with the sensor assembly illustrated in FIG. 3, in accordance with the invention;

FIG. 6 illustrates one embodiment of a current sensor which may be utilized in accordance with the invention, comprising a Hall effect current sensor;

FIG. 7 is a perspective and partially exploded view of an active support assembly in accordance with the invention, and expressly showing an array of flexible elements, an array of ring-shaped EAP actuators, and a mask for purposes of pre-straining EAP actuators;

FIG. 8 is a perspective and partially exploded view similar to FIG. 7 but showing the active support surface in accordance with the invention following an assembly of the elements shown in FIG. 7, and further showing the relative positioning of a thin foam layer above the active support surface;

FIG. 9 is a perspective and partially exploded view of a further embodiment of an active support assembly in accordance with the invention, with the assembly including an array of electroactive polymer push-pull actuators having stationary ends fixedly attached to the frame or subframe, along with movable ends;

FIG. 10 is a diagrammatic view showing the relative displacement of a bottom EAP actuator in a push-pull configuration, and specifically showing the concept of loading decreasing the displacement of the actuator until resistive forces of the actuator balance the load;

FIG. 11 is a two-dimensional diagram illustrating the relationship of displacement versus applied forces, expressly showing that as a magnitude of an externally applied force or load will increase, the displacement of the EAP actuator decreases while its resistive forces increase for an equivalent activation signal;

FIG. 12 is a schematic diagram of a conventional RC circuit, where a resistor has been inserted in series with the EAP actuator;

FIG. 13 is a two dimensional diagram illustrating the relationship between current and time for the circuit illustrated in FIG. 12;

FIG. 14 is a diagram similar to FIG. 13, but showing integrated current versus time for the circuit of FIG. 12;

FIG. 15 is a diagram similar to FIGS. 13 and 14, but expressly showing the value of current over time for a two mm change in height;

FIG. 16 is a diagram similar to FIG. 15, but expressly showing the relationship between integrated current and time for the two mm change in height;

FIGS. 17 and 18 together comprise a diagrammatic and functional description of one embodiment of an active support assembly in accordance with the invention, capable of mimicking a passive surface, along with illustrating a block diagram of the associated electronics relating to the functionality of the support assembly; and

FIG. 19 is a representative view of a surface characterization assembly having an array of nodes, with each node being a rigid disk corresponding to an active device in the active support assembly in accordance with the invention.

### DETAILED DESCRIPTION OF PREFERRED EMBODIMENTS

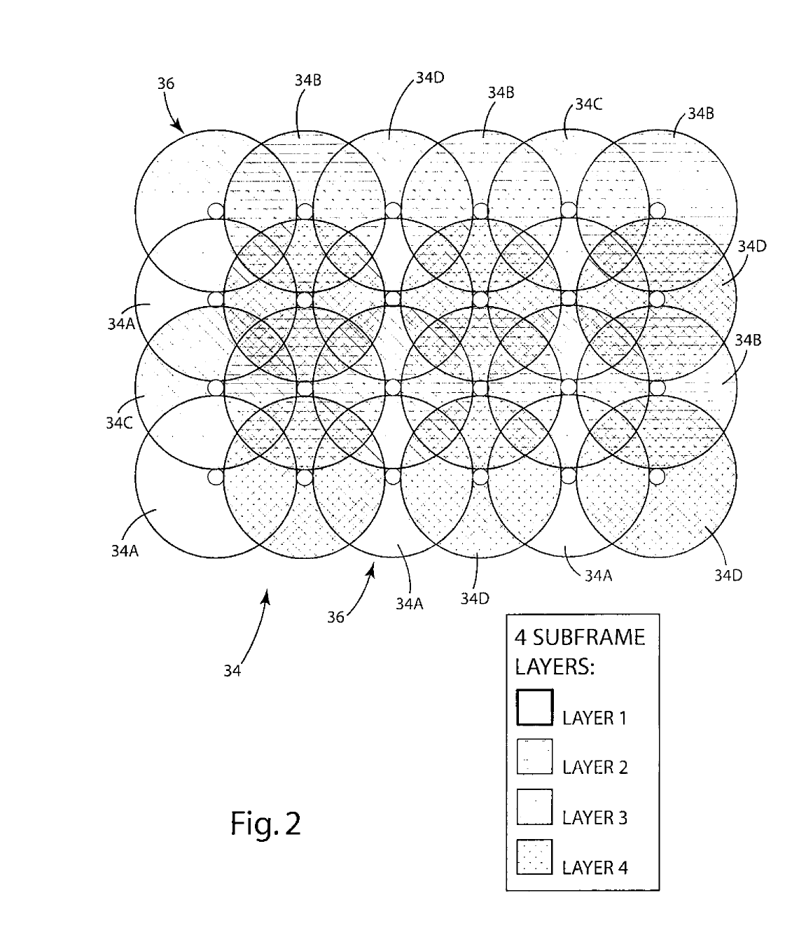

The principles of the invention are disclosed, by way of example, with respect to active support assemblies as described herein and illustrated primarily in FIGS. 2-19. More specifically, active support assemblies in accordance with the invention are described in detail herein with reference to a plurality of preferred embodiments as illustrated in the drawings. Numerous specific details are set forth herein for purposes of providing a thorough understanding of the present invention. However, it should be apparent to one skilled in the art that the present invention may be practiced without some or all of the specific details. That is, well known process steps and/or structural elements have not been described in detail, so as to not unnecessarily obscure the present invention.

As will be made apparent from subsequent description herein, the invention in part is comprised of a system for enhancing comfort and/or postural support afforded occupants of mattresses, overlays, and/or seating. In accordance with one concept of the invention, the system includes a plurality of active devices, with the devices configured so as to form a support surface. Controller elements are in operable communication with the active devices, so that the controller elements selectively apply activation signals to active material of the active devices, and thus effect changes in an attribute of the active material. These changes in the attribute result in desired changes in movement and resistive forces of the active devices, and in the shape and behavior of the support surface.

In addition, systems in accordance with the present invention can include elements for measuring changes in the attribute of the active materials, resulting from externally applied forces. Elements of the system are also included for purposes of providing the measurements to the controller elements so as to selectively apply the activation signals to the active material devices.

In accordance with other aspects of the invention, the system can include elements to isolate vibrational forces acting upon the occupants. The array of active support elements can form a plane of support, so that each of the elements is operable to move independently of other ones of the active elements. Elements are also provided for initializing positions and velocities of the active support elements. The invention can also include the continuous sensing of movements of the support elements resulting from the application of external forces. Computing elements are also included so as to compute magnitudes of the applied external forces, based upon the sensed movements. The activation signals can be applied to the active support elements, based upon the computed changes in magnitudes of the applied external forces. In this manner, the activation signals can effect changes of positions of velocities of the active support elements and in the magnitudes of resistive forces of the active support elements. Advantageously, systems in accordance with certain aspects of the invention include means for controlling the changes in positions to within one micron, even with the changes in positions occurring with velocities of up to 12 meters per second. Still further, the changes in the velocities can occur at accelerations of up to at least 7,200 meters per second². Still further, audible sound produced by the changes in the position and velocities can be controlled so as to be less than one db(A).

In accordance with other aspects of the invention, the invention can include a frame assembly, with a series of support plates. An array of active devices can also be provided, with the devices having stationary ends fixedly attached to the frame assembly. Movable ends are operatively attached to the support plates.

The active devices can include electroactive polymer (EAP) actuators. A first section of each EAP actuator can be attached to a stationary end of the active device, and a second end of each EAP actuator can be attached to the movable end of the active device. Still further in accordance with certain aspects of the invention, each of the active devices can function as a sensor. Specifically, movement of the movable end of each of the active devices resulting from the application of an external force to the support plate effects a change in an attribute of the EAP actuator. The change in the attribute is measurable.

In accordance with further aspects of the invention, each of the active devices can function as an actuator. Application of an activation signal to the EAP actuator can effect a change in a further attribute of the EAP actuator. The change in the further attribute forcibly moves the movable end of the active device and the attached support plate.

In addition to the array of active devices, active support assemblies in accordance with the invention can include a controller in operative communication with each of the EAP actuators of each of the active devices. The controller continuously measures the change in the attribute of the EAP actuator, resulting from movement of the movable end of the active device and the attached support plate.

More specifically, and in accordance with other aspects of the invention, the controller can use a measured change in the attribute so as to compute a change in acceleration, velocity, and position of the movable end, as well as in the magnitude of the applied force. A controller uses the change in acceleration, velocity, and position of the movable end and the magnitude of the applied force so as to selectively apply an activation signal to the EAP actuator. The application of the activation signal effects a change in the attribute of the EAP actuator. The change in the attribute results in a desired change in the acceleration, velocity, and position of the movable end of the active device, and the attached support plate, as well as in the magnitude of the resistive force.

The foregoing is a brief summary of certain concepts of the invention and embodiments are disclosed in the subsequent description herein. Other inventive concepts will also be made apparent from the description.

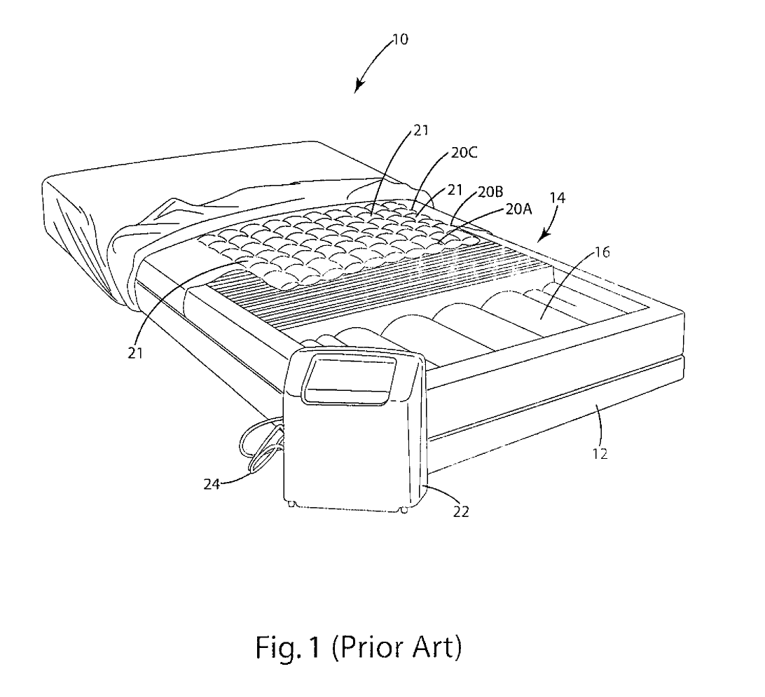

A substantial amount of background information regarding active support assemblies was previously set forth in the section entitled “Background Art.” As a further description of exemplary prior art, a known alternating pressure mattress system 10 is illustrated in FIG. 1. As shown therein, the mattress system 10 includes a base 12 which may be rigid in structure and supported on a ground level or on any suitable surface. Positioned on the base 12 is a pressure mattress 14. The pressure mattress 14 can include a head portion 16, on which an occupant may rest his or her head. Positioned below the head portion 16 is a cell portion 18. The cell portion 18 includes what can be characterized as a set of cell rows 20, with each cell row 20 extending transversely across the pressure mattress 14. Individual cell rows 20 are shown by example with the numerical references 20a, 20b, 20c, and . . . 20g, etc. depending upon the total number of cell rows 20. Each of the cell rows 20 includes a plurality of cells 21. Each of the cells 21 can consist of a flexible membrane. The flexible membranes or cells 21 can be maintained at varying air pressures as desired by the user. For purposes of inflation and deflation of these cells 21, an air vacuum/pump 22 can be attached to a pneumatic network (not shown) of air pipes (not shown) through an air hose 24. The air pipes (not shown) can include various valves which will be under control of the user for purposes of selective inflation and deflation of the cells 21. Although not shown in FIG. 1, the known mattress system 10 can also include a basic controller or similar means for purposes of selectively controlling the inflation/deflation of the cells 21 through the use of the vacuum/pump 22. In this manner, by varying the air pressure of the cells 21, the forces exerted on the various body parts of an occupant of the pressure mattress 14 can be varied as desired by the user.

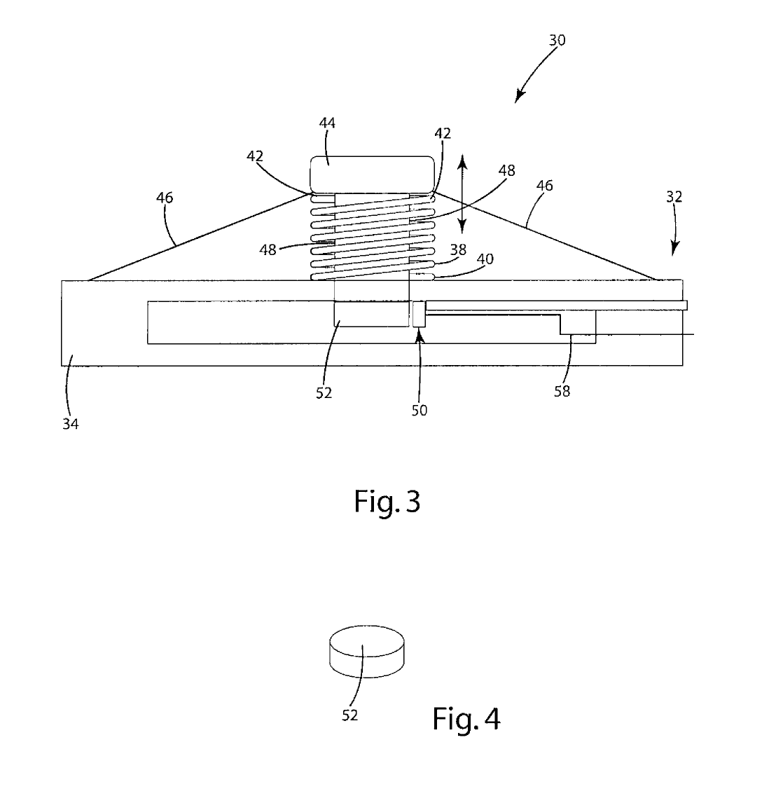

Turning to the specific embodiments of active support assemblies in accordance with the invention, basic elements of an active support assembly 30 are illustrated in a diagrammatic view as illustrated in FIG. 3. As shown therein, the active support assembly 30 (while showing only one active device) can consist of a frame assembly 32 which may have a rigid or semi-rigid configuration. The “rigidity” of an element such as the frame assembly 32 is often referred to by reference to the “compliance” of the element. That is, as the rigidity of an element decreases, the compliance of the element is said to increase. In this regard, the compliance of the frame assembly 32 should be sufficiently low so as to prevent distortion which may result from operation of the active devices of the support assembly 30. The frame assembly 32 shown in FIG. 3 may actually comprise two or more subframes 34. It should also be mentioned that the subframes 34, when associated with their active devices, can be characterized as “layers” or “layered subframes.”

In accordance with certain aspects of the invention, and with respect to layered subframes and rigidity of the same, a relatively rigid layer 34 may be paired with a compliant or semi-rigid layer 34. The compliant layer can be configured so as to comply with relatively larger and slower forces generated externally by the occupant or subject using the active support assembly. Correspondingly, the compliant combination of the rigid layer and the compliant layer will resist the relatively smaller and faster forces generated internally by operation of the active devices. Adding layered subframes 34 of electroactive polymer to an active device for purposes of increasing the level of forces that the active device is capable of exerting will correspondingly result in an increase in the size of the active device. If a coplanar arrangement of the active devices is assumed, it can be problematic to achieve a relatively high density of support plates as the size of the devices increase. Unfortunately, this relatively high density of support plates is necessary for purposes of achieving precision which is essentially demanded of the active support assembly 30 if the support assembly is to solve and substantially reduce the problems associated with the prior art.

However, an embodiment of active devices and subframes 34 which may be utilized in accordance with certain other aspects of the invention is illustrated in FIG. 2. As shown therein, the active devices 34 are arranged in layered subframes. With reference to FIG. 2, the active devices are symbolically illustrated as active devices 36. The layered subframes 34 consist of four layers, identified as layers 34a, 34b, 34c and 34d. With a layered subframe configuration as illustrated in FIG. 2, an arrangement of the active devices 36 can be achieved which essentially increases the depth of the active support assembly 30. Still further, a relatively higher density of active devices 36 (and, correspondingly, the support plates) is achieved, even though the size of the active devices 36 may increase.

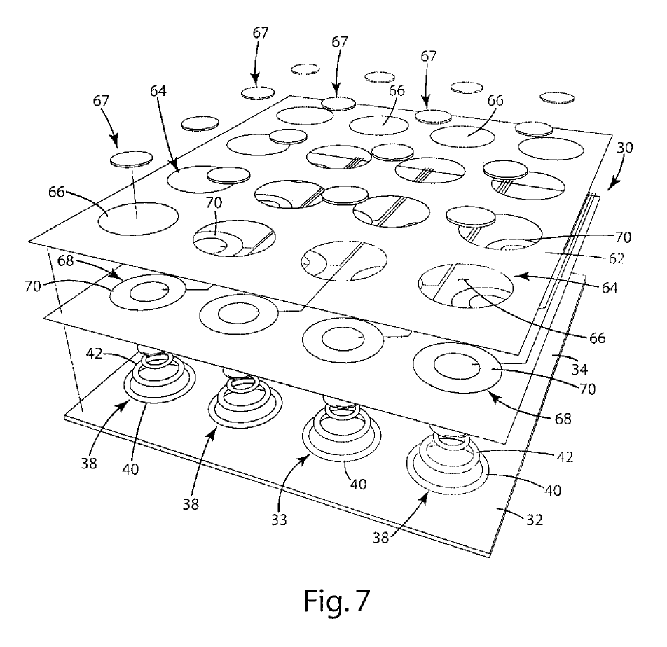

Reference is now made to FIGS. 3 and 7 with respect to the construction of the active devices 36. As shown in these drawings, each of the active devices 36 includes a flexible element 38. Each of the flexible elements 38 can be characterized as an “energy storage and return” element. These flexible elements 38 may take various forms, without departing from the principal novel concepts of the invention. For example, as shown in FIGS. 3, 7 and 8, each of the flexible elements 38 may be in the form of a spring formed and constructed so as to be under tension when assembled with the active device 36. Alternatively, in place of springs 38, the flexible elements may consist of elements such as air-filled cells, gel-fill cells, fluid-filled cells, foam or a combination of two or more of the forgoing. In the particular embodiment described herein, the flexible elements 38 may have a cylindrical or conical shape. However, other shapes of flexible elements 38 may also be utilized. Correspondingly, the radii of the flexible elements may be equal or not equal. The force constants of the flexible elements 38 may also be equal or non-equal, as well as their lengths. As a general rule, it is preferable for the choice of a force constant to be based on the desired response of the particular flexible element 38 to its maximum expected load.

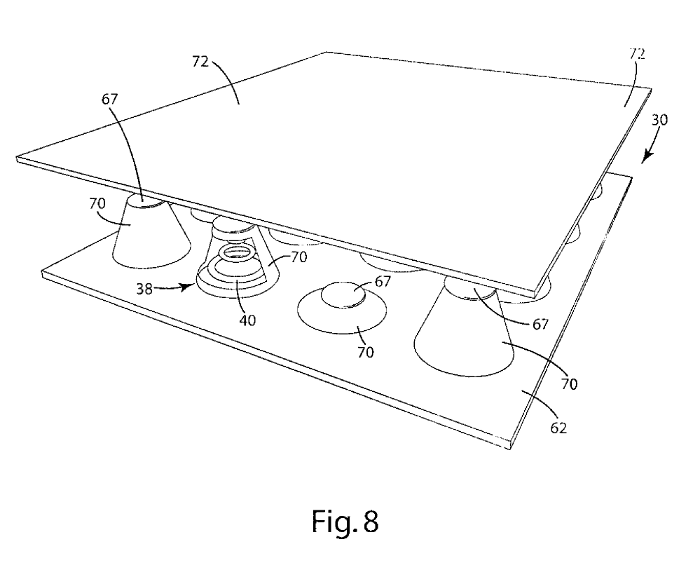

As apparent from the embodiment of the flexible elements 38 as illustrated in FIGS. 7 and 8, the centers of any two adjacent flexible elements 38 are separated by a distance equal to or larger than the sum of the maximum radii of the two flexible elements 38. With reference again to FIGS. 2 and 7, each of the flexible elements 38 can be characterized as having a stationary end 40. Each of the stationary ends 40 can be fixedly attached to the frame assembly 32 or subframe 34. Correspondingly, the opposing end of each of the flexible elements 38 can be characterized as comprising a movable end 42. With the flexible elements 38 being springs as illustrated in FIGS. 3 and 7, the moveable ends 42 can further be characterized as being directly connected to the stationary ends 40, but moveable relative to the stationary ends 40. In the configuration illustrated in FIGS. 3 and 7, each of the moveable ends 42 is operatively attached to an output cap or disk 44. As will be described in greater detail in subsequent paragraphs herein, each of the flexible elements 38 can be further characterized as functioning so as to bias both the direction of movement and the resistive forces exerted by the corresponding EAP.

It should be noted that the active support assemblies in accordance with the invention as described herein utilize electroactive polymer or “EAP” actuators. However, it should be emphasized that it may be possible to utilize other types of devices having attributes similar in function to EAP actuators, without departing from certain concepts of the invention. In any event, the subsequent disclosure herein will be directed to active support assemblies employing EAP actuators.

General principles associated with the structure and operation of EAP actuators were previously described herein in the section titled “Background Art.” As used with active support assemblies in accordance with the invention, EAP actuators may have flexible electrodes printed on top and bottom surfaces. Such electrodes may be aligned with one another, and may also have substantially identical sizes and shapes. For purposes of functioning of the EAP actuators, an activation signal may be applied to the actuator. In response to the activation signal, an EAP actuator will expand or contract. This action of expansion and contraction can be employed with active support assemblies in accordance with the invention for purposes of producing forces, displacement or a combination of the two. Further, and as previously explained in part with respect to FIG. 2, two or more layers of EAP actuators may be combined, and will result in an increase in the forces exerted by the active devices employing the EAP actuators.

Still further, an EAP actuator can essentially be characterized as a flexible capacitor. In its basic form, a capacitor can be characterized as a passive electrical component for storing energy in an electrical field between a pair of conductors or “plates.” Although most capacitors are designed so as to maintain a fixed physical structure, structures which can be changed as a result of various factors can result in changes in capacitance. These resulting changes in capacitance can be utilized to sense the attributing factors. In this regard, external forces applied to an EAP actuator can result in deformation of the actuator. This resultant deformation correspondingly produces a change in the capacitance of the EAP actuator. This capacitance change can be measured through various processes. For example, the change in capacitance can be measured by measuring the change in voltage across the EAP actuator electrodes. Correspondingly, another procedure for measuring this capacitance change may include the measurement of the amount of current drawn by the actuator. However, it should be mentioned that current measurements require an assumption of an approximate “ideal” voltage source.

The electroactive polymer elements which may be used with active support assemblies in accordance with the invention may consist of various types of materials. In general, materials suitable for use as an electroactive polymer with the present invention may include any substantially insulating polymer or rubber (or combination thereof) that deforms in response to an electrostatic force. Correspondingly, suitable materials may be used which result in a change in an electric field upon deformation. Other materials which may also be utilized for use as electroactive polymers with the present invention include pre-strained polymers, which may include silicone elastomers, acrylic elastomers, polyurethanes, thermoplastic elastomers, copolymers comprising PVDF, pressure-sensitive adhesives, fluoroelastomers, polymers comprising silicone, acrylic moyeties, and the like.

The active support assemblies 30 can include a series of support plates 46 which are utilized in combination with the frame assembly 32. The support plates 46 can be correspondingly and pivotably mounted to corresponding active devices 36. More specifically, the support plates 46 can be pivotably mounted to output caps 44 or output shafts (described subsequently herein) 48. This mounting can occur through the use of any desirable means, and may or may not include intervening extension rods. The support plates 46 may have a circular, square or other shape with respect to their configurations. Also, the radii of the support plates 46 may be equal to or larger than the radii of the corresponding active devices 36 to which the support plates 46 are mounted. Preferably, the support plates 46 may pivot about their centers. This pivoting capability accommodates unbalanced loads. In this regard, it is preferable for the support plates 46 to be “pre-loaded,” so as to assure that the support plates 46 have positions orthogonal to the lengths of the corresponding active devices 36, when the plates 46 are unloaded. Still further, it is advantageous for the support plates 46 to be composed of rigid or compliant (i.e., semi-rigid) material. The support plates 46 may also be preferably covered with a thin layer of foam or other compliant material. In general, the support plates 46 can be characterized as forming one or more planes of support, when the corresponding active devices 36 of the support plates 46 are driven by certain activation signals.

Turning to other elements of the active support assembly 30, and with reference to FIGS. 3-8, the output caps or output disks 44 associated with each active device are attached to the corresponding output shafts 48. These output shafts 48 can also be characterized as position sensing shafts 48. The position sensing shafts 48 essentially extend away from the support surface which is formed by the support plates 46. In fact, and as shown in FIG. 3, the position sensing shafts 48 may partially penetrate the frame assembly 32 or a subframe 34. The position sensing shafts 48 are configured and essentially function so as to sense the positions of the attached output disks or output caps 44. Through the sensing of the positions of the output caps 44, the shafts 48 also operate so as to sense the positions of the attached support plates 46. It should be emphasized that there are several possible position sensing configurations which may be utilized with active support assemblies in accordance with the invention. For example, each of the possible sensing configurations may utilize a sensor 50. The sensor 50 may be attached to the position sensing shaft 48 or, alternatively, to a frame assembly 32 or subframe 34.

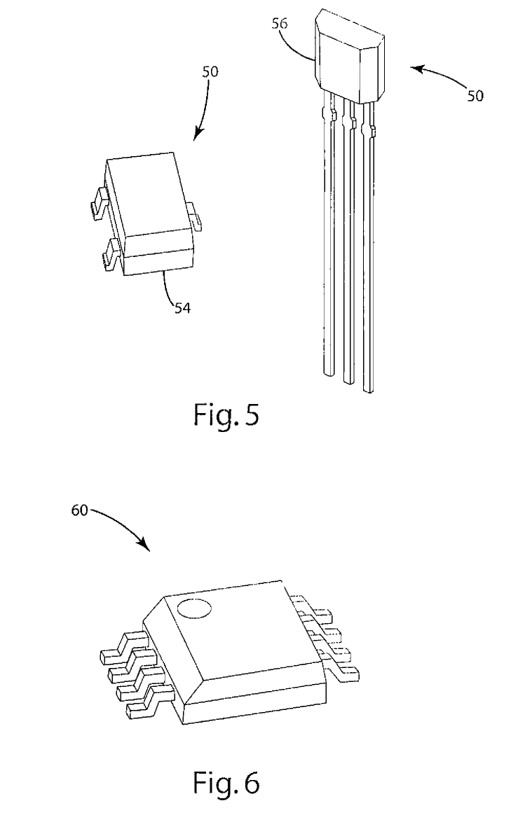

One specific sensing configuration is illustrated in the cross-sectional view of FIG. 3. As shown therein, a permanent magnet 52 (particularly shown in FIGS. 3 and 4) can be mounted on the end of the position sensing shaft 48. The magnet 52 can be any of a number of different types of permanent magnets. For example, one type of permanent magnet which may be utilized is a 26 MGOe, Samarium Cobalt Disc Magnet, preferably having a diameter of 0.375 inches and a thickness of 0.125 inches. The magnet is identified as SMCO-D1, suitable for high temperature applications and having a rating of 10,600 gauss. In addition to the permanent magnet 52, FIG. 3 illustrates the use of a linear Hall effect switch or sensor 50 which may be attached to the frame assembly 32 or a subframe 34. FIG. 5 illustrates two versions of Hall effect switches which may be utilized as the switch 50. More specifically, FIG. 5 illustrates a switch 54 which may be characterized as a 3-pin SOT switch, while switch 56 represents a 3-pin SIP switch. The 3-pin SOT switch can be one which is manufactured by Allegro Microsystems, and identified as part number A1145LLHLT-T, identified as a Hall effect IC switch, having a Digi-Key part number of A1145LLHLT-T-ND.

The length of the position sensing shaft 48 and the locations of the permanent magnet 52 and the Hall effect switch 50 may be preferably chosen so that substantially the maximum magnetic flux density occurs when the output cap 44 of the active device 36 is in what can be characterized as a neutral or zero position. The Hall effect switch 50 can further be configured so as to output values as follows: A logical “1” when the magnetic flux density exceeds a threshold associated with the zero position; and a logical “0” otherwise. In this regard, the output signal from the Hall effect switch 50 as illustrated in FIG. 3 may be produced on line 58 and applied to various other components of the support assembly as appropriate.

A controller (to be described subsequently herein) may either sample the output signal on line 58 or, alternatively, the signal on line 58 may be routed to an interrupt pin or similar element on the controller for purposes of obtaining an immediate response when a signal change is generated on line 58. Still further, the zero position signal may be utilized for purposes of correcting for “drift” in position measurements that may occur when such measurements are based solely on the change in EAP actuator capacitance.

As an alternative, the linear Hall effect switch 50 may be replaced by a linear Hall effect sensor 60. An example linear Hall effect sensor 60 which may be utilized in accordance with the invention is illustrated in FIG. 6. This Hall effect sensor 60 may be one manufactured by Allegro Microsystems. The sensor is identified as an IC Hall effect sensor BIP 8-SOIC, having a part number of ACS712ELCTR-20A-T. The Digi-Key part number is 620-1190-2-ND. With the replacement by the linear Hall effect sensor 60, the sensor 60 can be configured so as to generate an output signal representative of the change in the magnetic flux density. This change in flux density will be proportional to the change in position of the permanent magnet 52 with respect to the sensor 60. In this manner, the controller can obtain requisite data by continuously sampling the position of the output disk or cap 44.

Although not specifically shown in the drawings, it should be emphasized that other embodiments of active support assemblies in accordance with the invention may be constructed using various other means for performance of position sensing processes. For example, position sensing may be accomplished through the use of an accelerometer for measurement of acceleration over time. With such measurement, velocity and displacement can be derived through conventional computations. If an accelerometer is utilized in accordance with the invention, such an accelerometer may be directly mounted to the position sensing shaft 48. In still another embodiment, the function of position sensing may be accomplished through the use of switching means incorporating an electromechanical switch. Such a switch, for example, may be a two stage switch. That is, the electromechanical switch may be in an “on” state when the position sensing shaft is in what can be characterized as a “neutral” position. The switch may otherwise be in an “off” state.

In a still further embodiment, the position sensing function may be accomplished through the use of an LED or laser diode, in combination with a photodetector. In such an embodiment, the position sensing shaft 48 may permit light from the LED or laser diode to pass through in a manner so as to be detected by the photodetector only when the position sensing shaft 48 is in the neutral position.

As previously referenced herein, the active support assemblies in accordance with the invention may also employ a controller for control and communications with each of the active devices 36 associated with the active support assembly 30. More specifically, the controller can be utilized to transmit and receive signals to and from each active device, respectively, in an array of active devices. The communications can occur through the use of one or more multiplexors. In this regard, a wiring harness or similar device can be utilized to connect the multiplexors to each of the EAP actuators in each active device 36 of the array. In one embodiment, the wiring harness can provide at least two connections to each EAP actuator. One of these connections can be utilized by the controller to supply an activation signal to a desired EAP actuator. The second connection can be utilized for purposes of the controller (through the multiplexors and wiring harness) receiving signals from the sensor. The sensor may be one which is configured so as to measure the change in capacitance of the EAP actuators caused by deformation. An example of such a sensor can comprise a current sensor which senses the electrical current drawn by the EAP actuator. As previously described herein, FIG. 6 illustrates an appropriate linear Hall effect sensor 60 which may be utilized for this purpose. In addition to the two connections between the wiring harness and each EAP actuator, a third connection can also be made from the appropriate multiplexor to the active device through the corresponding wiring harness, for purposes of enabling the controller to receive position signals from the EAP actuator.

In a physically realized embodiment of a support assembly in accordance with the invention, the controller can operate the multiplexor to selectively read the sensor signals from each active device 36 in sequence. In this regard, the controller can operate an analog-to-digital convertor so as to sample and convert the analog sensor signals to digital sample data. For this function, the controller can employ a first algorithm which uses the digital sample data so as to derive changes in position, velocity and/or acceleration of the moveable end of each active device 36. Still further, the controller can employ a second algorithm which utilizes a change in position, velocity and/or acceleration of the moveable end of each active device 36 so as to derive a change in magnitude of the external forces applied to the moveable end of the device 36. Following these functional operations, the controller can further apply a third algorithm which uses the change in position and the change in the magnitude of the externally applied forces, so as to compute a new position, velocity and/or acceleration for the moveable end of each active device, along with a new magnitude of the resistive force generated by the corresponding active device 36. As an optional function, the controller can still apply a fourth algorithm which uses the changes in magnitudes of the externally applied forces on the moveable ends of all active devices 36, so as to compute a series of new positions, velocities and/or accelerations for the moveable ends of the active devices, along with a series of new magnitudes for the resistive forces of the active devices.

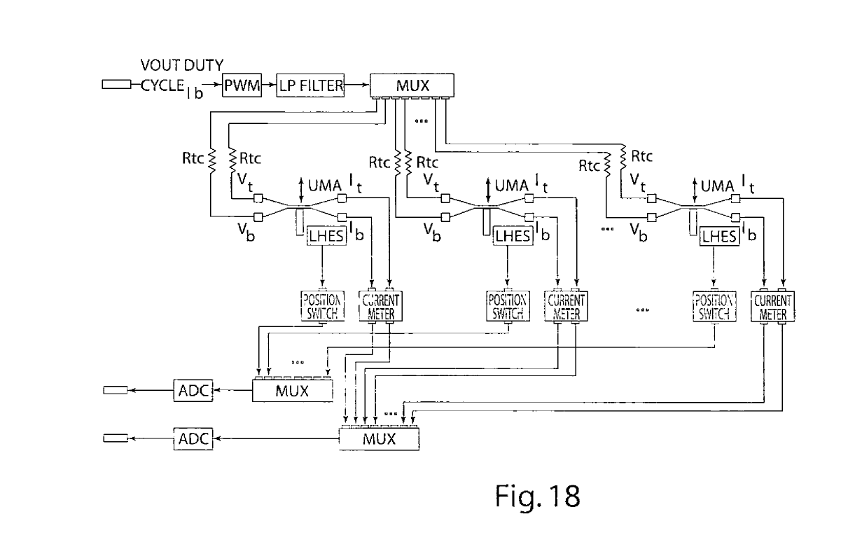

The functional operation of the controller can then further include the use of principles of superposition. In this regard, the controller can functionally operate so as to combine the series of new positions, velocities and/or accelerations for the moveable ends so as to obtain a single new position, velocity and/or acceleration for each moveable end of an active device 36, and combine the series of new magnitudes associated with the resistive forces of the active device, so as to obtain a new single magnitude for the derived resistive force. In addition to the foregoing, the controller can also employ a fifth algorithm which uses the electrical current and the new positions, velocities and/or accelerations for the moveable end of each active device, and the electrical current and new magnitudes for the resistive forces generated by the active device (along with a current activation signal), for purposes of deriving a new activation signal. The controller can operate the multiplexor so as to selectively apply the activation signals to each of the active devices 36, in sequence. The circuit configuration for operation of the controller with associated components will be described in greater detail herein with respect to FIG. 18.

As earlier illustrated and briefly described, one embodiment of an active support assembly 30 in accordance with the invention is illustrated in FIGS. 7 and 8. As shown therein, the active support assembly 30 includes the frame assembly 32. The frame assembly 32 can include an array 33 of flexible elements 38. As previously described briefly herein, each of the flexible elements 38 can include a stationary end 40 mounted directly to the frame assembly 32. As also previously described herein, the frame assembly 32 may also consist of one or more individual subframes 34. Each of the flexible elements 38 can include not only a stationary end 40, but also a moveable end 42, as also particularly shown in FIG. 7.

In addition to the foregoing elements, the active support assembly 30 can also include a mask element 62 having an array 64 of apertures 66 positioned therein. The mask 62 can be utilized for purposes of exerting what could be characterized as “pre-strain” forces on the EAP actuators. Still further, the active support assembly 30 includes an array 68 of ring-shaped EAP actuators 70. The array 68 (which can also be characterized as an EAP section 68) can include a single section of material having a plurality of EAP actuators 70 mounted thereto. The EAP actuator array 68 lies between the mask 62 and the array 33 of flexible elements 38. In this structure, the array 68 of the EAP actuators 70 is secured to the frame assembly 32 or subframe 34 by means of the mask 62. That is, the mask 62 is lowered over the array 33 of flexible elements 38 in a manner so that each EAP actuator 70 is stretched over a corresponding flexible element 38.

Still further, the active support assembly 30, as also briefly described previously herein, include an array 67 of output caps or disks 44 which can be attached to the moveable ends 42 of the flexible elements 38. It should also be noted that as an optional structural configuration, it would be possible to employ a series of extension rods. In addition, a series of support plates could be attached to the output caps 44, with or without intervening extension rods. Still further, a wiring harness can be employed, with a controller connected to the EAP actuators 70 through the wiring harness. In a physically realized system, the controller can be utilized to continuously measure the EAP actuator capacitances, and compute the support plate accelerations, velocities and positions. In addition, the controller can be utilized to derive the magnitudes of the externally applied forces on the support plates, and compute the desired support plate position based on the forces. Still further, and as earlier described, activation signals can then be translated to the EAP actuators, so as to mobilize the actuators 70 and move the attached support plates to the desired positions.

The EAP section 68 includes a single section of material, and incorporates a series of EAP actuators 70 organized into the array 68. The configuration is such that each of the actuators 70 is aligned with a flexible element 38 within the flexible element array 33. In the particular configuration illustrated in FIGS. 7 and 8, the electrodes of the EAP actuators 70 have a ring-shaped configuration. The inner radius of each ring is substantially equal to the radius of the movable end 42 of the corresponding flexible element 38. Correspondingly, the outer radius of each ring should be equal to or larger than the radius of the stationary end 40 of the corresponding flexible element 38. In selection of the appropriate materials for the EAP actuators 70, the materials should be chosen based on the force constant of the corresponding flexible element 38. That is, in the absence of an activation signal, or for a nominally low activation signal, the EAP actuators 70 should each compress its corresponding flexible element 38 by a desired amount. An increase in the level of the activation signal should cause the corresponding EAP actuator 70 to expand, and forceably extend the moveable end 42 and the corresponding output cap 44, with expansion of the flexible element 38. Correspondingly, a decrease in the activation signal level should cause the EAP actuator 70 to contract, compressing the flexible element 38 and forceably retracting the moveable end 42 and the output cap 44.

Turning again to FIG. 7, the mask 62, as earlier described herein, includes a series of apertures 66. The apertures 66 are concentric with and aligned with the flexible elements 38 of the array 33. The radius of each aperture 66 should be made equal to or larger than the radius of the corresponding flexible element 38. During assembly, the mask 62 is lowered and bonded to the frame assembly 32 or subframe 34. The positioning and the bonding occur in a manner so that the EAP actuators 70 which lie between the mask 62 and the frame assembly 32 or subframe 34 are stretched or “pre-strained” over the flexible element 38. After bonding has occurred, the mask 62 will not change the compliance of the frame assembly 32 or subframe 34.

With reference to the output caps 44, which were briefly described previously herein, the caps 44 are preferably rigid and mounted on the movable ends 42 of the flexible elements 38. The mounting occurs so that the inner, non-active centers of the ring-shaped EAP electrode pairs lie between the output caps 44 and the flexible elements 38. By constraining the active portion of the EAP (that is, the EAP actuators 70) between the rigid, stationary frame assembly 32 or subframe 34, and the rigid output cap 44, and with biasing of the direction of movement with the flexible element 38, the expansion of the EAP actuators 70 that occurs under application of an activation signal will correspondingly result in forceable movement of the output cap 44 away from the frame assembly 32 or subframe 34.

Other embodiments associated with the assembly and structural configuration of each of the active support assemblies 30 may be utilized, without departing from the principal novel concepts of the invention. For example, with respect to the structural assembly, the EAP array or section 68 may be bonded directly to the frame assembly 32 or subframe 34, in the absence of the use of a mask 62. For example, structural assembly could occur through the use of vacuum forming, and the use of an appropriate adhesive.

Still further, another embodiment of a support assembly 30 in accordance with the invention may include the EAP array or section 68 being configured as a “stand alone” device. That is, the EAP section 68 would be formed as a flexible overlay, without the use of a frame assembly 32, mask 62 or output caps 44. More specifically, multiple layers of the EAP material would be combined, and the EAP actuators 70 on each layer of the material could be aligned and combined so as to increase the forces exerted by the EAP actuators 70. Correspondingly, the direction of the movement of the EAP actuators 70 could be biased by bonding the EAP section 68 to a compliant, monolithic substrate. The substrate can comprise an array of raised flexible elements. An example of such a configuration would be one utilizing a contoured foam layer.

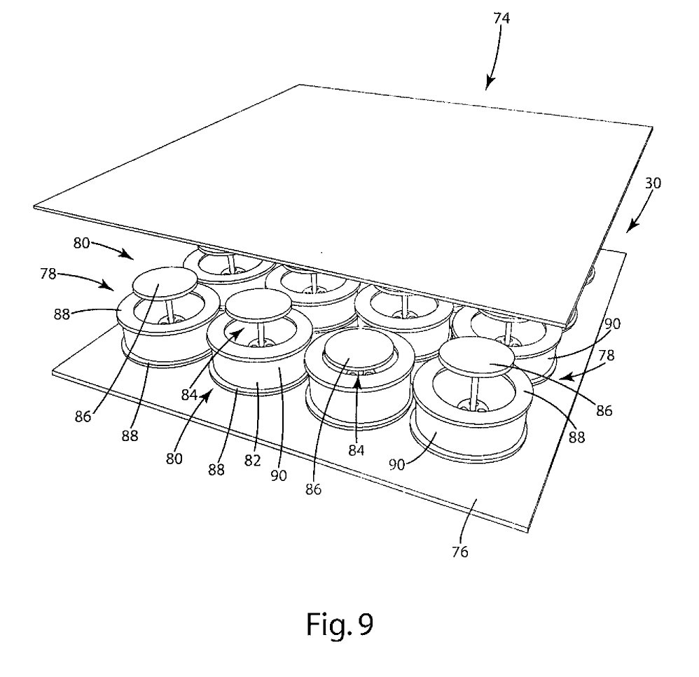

Another embodiment of an active support assembly 30 in accordance with the invention is illustrated as support assembly 74 in FIG. 9. With reference to FIG. 9, the active support assembly 74 includes a frame assembly 76, which can substantially correspond to the previously described frame assembly 32. An array 78 of EAP push-pull actuators 80 are mounted to the frame assembly 32. Specifically, the push-pull actuators 80 include stationary ends 82 which are fixedly attached to the frame assembly 76. Opposing the stationary ends 82 are a series of moveable ends 84. As with the active support assemblies 30, a set of extension rods can be optionally utilized. Attached to the moveable ends 84 of the array 78 of push-pull actuators 80 are a series of support plates 86. The attachment can occur with or without intervening extension rods. In addition to the foregoing elements, the alternative embodiment of the active support assemblies 74 can also include a wiring harness, with a controller connected to the EAP push-pull actuators 80 through the wiring harness. As with other EAP actuators described herein, the controller can continually measure the capacitances of the EAP push-pull actuators 80, and compute accelerations, velocities and positions of the support plates 86. Through these computations, the magnitudes of externally applied forces on the support plates 86 can be derived. Based on the derived magnitudes of the externally applied forces, desired support plate positions can be computed. Following such computation, activation signals can be transmitted to the EAP push-pull actuators 80, so as to mobilize the actuators 80 and move the attached support plates 86 to the desired positions.

With further reference to FIG. 9, each of the EAP push-pull actuators 80 can include a pair of EAP actuators 88 which can be suspended transversely between a pair of parallel outer frames and a center output disk. The outer frames can be separated longitudinally by a spacer 90, with each EAP actuator 88 capable of exerting either a pulling or a pushing force on the output disk. The directions and magnitudes of the forces exerted by the pair of actuators 88 are configured so as to forcefully move the output disk. That is, the output disk can be moved by the first actuator 88 pushing on the disk, while the second actuator 88 pulls on the disk. Conversely, a configuration can be provided whereby the first actuator 88 will pull on the disk, while the second actuator 88 will exert pushing forces on the disk. With the active portion of the electroactive polymer (i.e. the EAP actuator 88) being constrained between a rigid outer frame and the rigid center output disk, expansion of the EAP actuator 88 that occurs under application of an activation signal will necessarily result in forceful movement of the center output disk away from the rigid outer frame. Either or both of the frames may be attached to the frame assembly or subframes 76. Two or more of the EAP push-pull actuators 80 may be stacked together so as to increase displacements produced by the active support assembly 74. It should be noted that either or both of the outer frames of a stacked set of actuators 88 may be attached to the frame assembly or subframe 76, or the outer frames of adjacent actuators. The output disk can be attached to an output shaft, where the shaft communicates the motion of the output disk to a support plate or thin foam layer 72, with or without the use of intervening extension rods.

It should be noted that forces are exerted by the EAP actuators 88 during contraction. Further, contractions are triggered by a decrease in activation signal levels and used in the embodiments described herein for purposes of generating pulling forces. In view of the foregoing, the pulling forces may be somewhat less than the forces exerted by the EAP actuators 88 during expansion, in that expansion is triggered by an increase in the activation signal level and is used in the embodiments described herein to generate pushing forces. In view of all of the foregoing, push-pull configurations incorporating a pair of EAP actuators 88 may therefore provide advantages over single EAP actuator configurations. That is, the pair of EAP actuators 88 in a push-pull actuator 80 configuration are capable of exerting substantially equal forces on the output disk in either direction. This occurs because one of the EAP actuators 88 is configured to exert pushing forces, while the other actuator 88 is configured so as to exert pulling forces on the output disk. These potential performance differences between the configuration of push-pull actuators 80 and single configuration EAP actuators 88 may become particularly apparent when an external load (i.e. an external force) is applied to the active device, where the load is substantially less than the load limit of the actual device. In such a situation, a loaded push-pull actuator 80 configuration would be expected to generate rapid acceleration in either direction. Conversely, a loaded single configuration of EAP actuators 88 would be expected to generate rapid acceleration only in one direction; namely, the direction associated with expansion of the EAP actuator 88. That is, rapid acceleration may not be expected in the opposing direction, with the opposing direction being associated with contraction of the EAP actuator 88. Dependent upon the configuration and the force constant of the flexible element, acceleration in the direction associated with contraction of the EAP actuator 88 may be further degraded in that some fraction of the total force of retraction may be required so as to overcome resistive forces of the flexible element. This imbalance and acceleration may limit the ability of active support assemblies based on single EAP actuator 88 configurations to effectively mimic passive support services.

The immediately following paragraphs of this disclosure will now discuss general principles associated with processing directed to pressure mapping for use of the active support assemblies in accordance with certain concepts of the invention. For purposes of describing these pressure mapping functional sequences, reference is first made to a description of concepts associated with a capacitor and capacitance. Certain of these concepts were previously described herein in the section entitled “Background Art.” However, some of these principles will again be repeated. More specifically, a capacitor is an electrical/electronic device capable of storing energy in the electric field between a pair of conductors. These conductors are typically referred to as “plates.” Equation 1 below defines the capacitance C (often referred to as the “charge-storing capacity”) of a device as follows:

C=Es*(A/d) (Equation 1)

where Es is the static permittivity, A is the surface area of each plate, and d is the separation distance of the plates.

Equation 2 below relates the current, voltage and charge quantity on the capacitor plates as follows:

i=dQ/dt=C*dV/dt (Equation 2)

where i is the current, dQ/dt is the rate at which charge flows into the device, C is the device capacitance, and dV/dt is the rate at which the voltage across the device changes.

An EAP actuator can be formed by laminating a thin dielectric (i.e. EAP) film with flexible electrodes. With a voltage potential applied across the electrodes, and through the principles of Maxwellia n forces, the electrodes will attract each other. The attraction will result in the corresponding forces of the film to contract in thickness and expand in area. When mechanical constraints and output linkages are fixed to the film, the expansion and contraction of the film can be essentially “harnessed”.

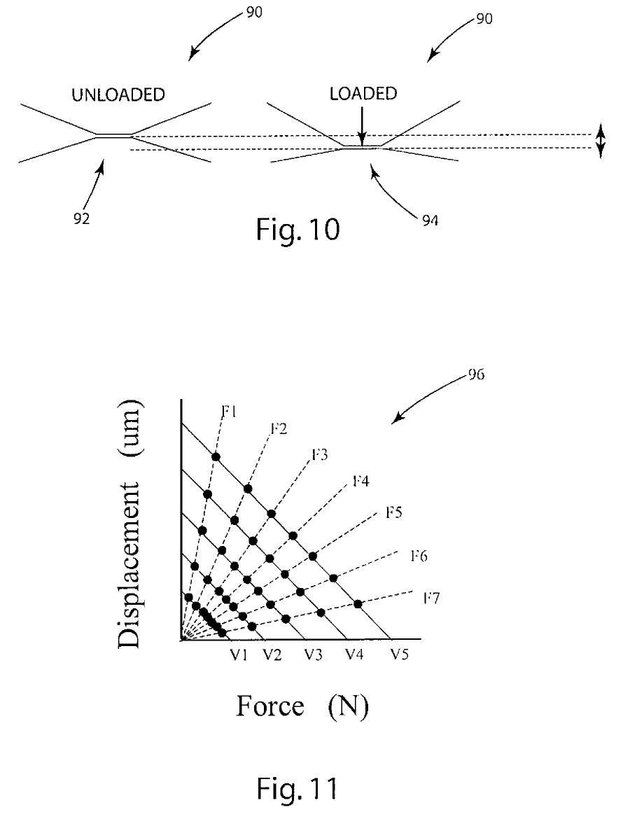

The capacitor configuration described in Equations 1 and 2 essentially relates to what is characterized as a “flexible” capacitor. In accordance with Equation 1, the capacitance of an EAP actuator will change as it undergoes deformation. More specifically, when the EAP actuator expands, as it will when the level of its activation signal is increased or the magnitude of applied force is reduced (assuming the direction of this force opposes expansion), its area (which can be defined as area A) becomes larger and the separation distance d becomes smaller. In this manner, capacitance is increased. Conversely, when the level of the activation signal for the EAP actuator is decreased or the magnitude of applied forces is increased, the EAP actuator will contract. That is, the area A will become smaller and the separation distance d will become larger. Correspondingly, the capacitance will be decreased. FIG. 10 illustrates a capacitor 90 and shows the effects of loading on the bottom of the EAP actuator in a push-pull configuration. More specifically, reference number 92 represents capacitance with the bottom of the EAP actuator unloaded, while reference numeral 94 illustrates the effects of loading on the bottom of the EAP actuator.

The forces exerted by the EAP push-pull actuators 80 decrease with displacement. Displacement can also be characterized as “stroke.” At maximum displacement, an EAP actuator will exert no force. Conversely, at zero displacement, the EAP actuator will exert its maximum force. When an external force (or load) is applied to the EAP actuator, the performance therefore changes. More specifically, as the magnitude of an externally applied force increases, the displacement of the EAP actuator will decrease and the resistive forces will increase for the same activation signal. The activation signal can be characterized as signal Va. FIG. 11 illustrates this general concept. More specifically, FIG. 11 is a graph 96 illustrating changes in displacement (in micrometers) versus resistive forces as shown by the activation signals V1 through V5. These forces would typically be represented by Newtons. Correspondingly, the applied forces in FIG. 11 are illustrated as applied forces F1 through F7. As shown in FIG. 11, with the applied forces increasing from F1 through F7, the displacement of the EAP actuator will decrease. Correspondingly, the resistive forces of the EAP actuator will increase for what can be characterized as the same activation signal. This configuration is somewhat similar to a spring, with a force constant being determined by the activation signal Va.

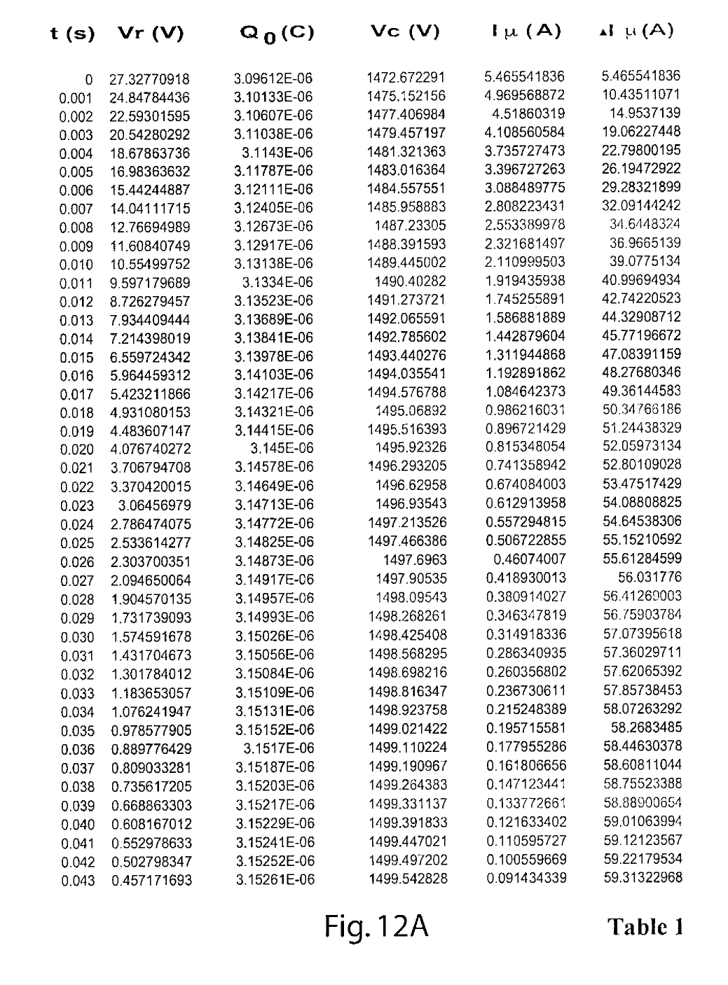

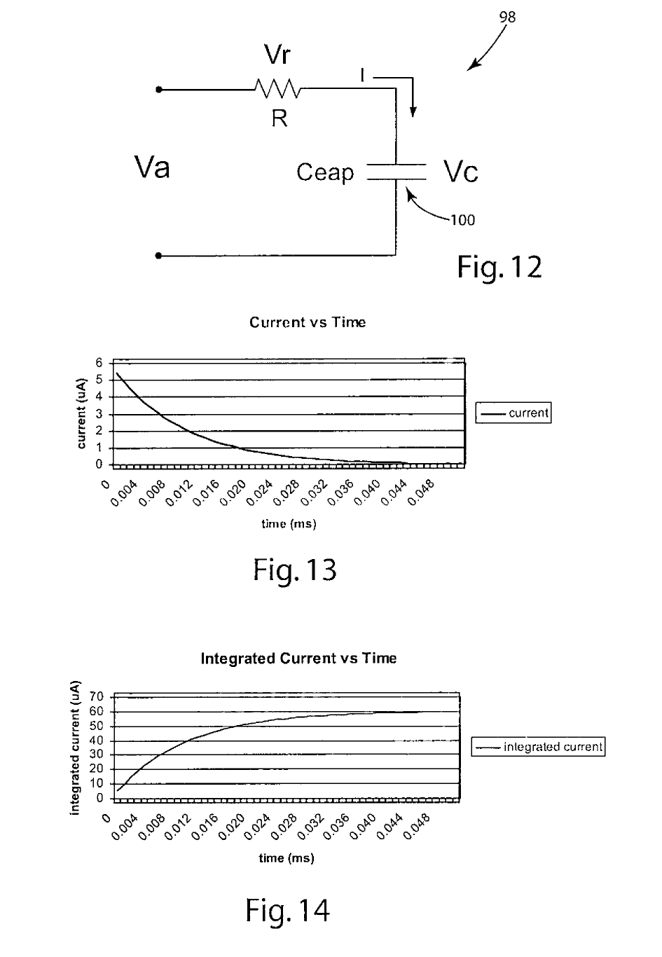

Turning again to the principles of the function of the EAP actuators and capacitance, FIG. 12 illustrates what is well known as an RC circuit 98. The RC circuit 98 includes a resistor R. The resistor R is positioned in series with an EAP actuator 100. The EAP actuator 100 is represented as Ceap. The voltage across the capacitor is represented as Vr, while the voltage across the EAP actuator is represented as Vc. In accordance with the foregoing, the total voltage across the RC circuit 98 is represented as voltage Va and is calculated in accordance with Equation 3 as follows:

Va=Vr+Vc (Equation 3)

Correspondingly, the current I running through the RC circuit 98 can be shown as being equivalent to the current Ir passing through the resistor R. This current also equates to the current Ic passing through the EAP actuator Ceap. Further, the time constant of the RC circuit 98 can be characterized as time constant TC. This time constant can be characterized as corresponding to the length of time that is required for the voltage Vc across the capacitor or EAP actuator Ceap to reach 1/e (or approximately 63%) of its final value. This time constant can be characterized in accordance with Equation 4 as follows:

TC=R*Ceap (Equation 4)

As earlier stated, the capacitance Ceap of an EAP actuator will increase when the EAP actuator is under the application of external forces. This is illustrated by the following Equation 5, which essentially is a rewrite of Equation 2:

Q=VC (Equation 5)

From Equation 5, it follows that as the capacitance Ceap increases, the voltage across the capacitor Vc is reduced, in view of the fact that the charge on the capacitor plates Q cannot instantaneously change. The current I through the RC circuit 98 will increase so as to charge the capacitor Ceap, and the voltage across the capacitor Vc will change in accordance with Equation 6 as follows:

Vc(t)=Vs*(1−et*P) (Equation 6)

In accordance with all of the foregoing, the capacitor therefore charges at a rate determined by the resistance R and the capacitance Ceap.

Referring again to Equation 1, it is apparent that the capacitance of the EAP actuator will depend on the permittivity of the dielectric film, the area of the plates and the separation distance. The permittivity of free space is approximately 8.854187817E-12 f/m. Correspondingly, the relative static permittivity, Cr, also referred to as the dielectric constant, of electroactive polymers will range from 2 to 12. This will correspond to a static permittivity, Cs, which ranges from 1.770837563E-11 f/m to 1.062502538E-10 f/m. In accordance with the foregoing, Equation 7 can be written as follows:

Es=Er*E0 (Equation 7)

The EAP actuators described in the foregoing embodiments can have a shape approximating that of a conical frustrum. The surface area S of the frustrum can be calculated using Equation 8 as follows:

Surface Area=n*(R1+R2)*sqrt[R1−R2)2+H2] (Equation 8)

where R1 is the radius of the top of the conical frustrum, R2 is the radius of the bottom of the conical frustrum, H is the height of the conical frustrum, and the areas of the top and bottom circles are not included.

As an example of the foregoing, an EAP actuator can be assumed having a height of 0.016 m, top radius of 0.025 m and bottom radius of 0.05 m. Such an actuator will have a surface area of 0.006994 m². Film thicknesses will typically range from 30 to 60 microns. Assuming a dielectric film thickness of 60 microns, the capacitance of the EAP actuator would range from 2.064080189E-09 f to 1.23844811E-08 f.

With reference again to the RC circuit 98 shown in FIG. 12, it can be assumed that the voltage across the RC circuit 98 Va is 1500 V. Correspondingly, the resistance R can be assumed to be 5Mc. Still further, a capacitance Ceap of the EAP actuator 100 can be assumed to be 2.064080189E-09 f. With the foregoing, and using Equation 6, the charge Q, on a fully charged capacitor Ceap would be 3.09612E-06 coulombs. Correspondingly, the voltage, Vc, would be approximately 1500 V. A 1 mm increase in the height of the conical frustrum will change the surface area of the EAP actuator to 0.007123 m² and its capacitance to 2.102382386E-09 f. After such a change in height, and specifically immediately thereafter, the charge, Q, on the capacitor Ceap would be the same, since the charge can not instantaneously change. The voltage Vc across the capacitor Ceap would drop from approximately 1500 V to 1472.672291 V. Correspondingly, and in accordance with prior calculations described herein, the voltage across the capacitor Ceap, Vc, would change as previously described. Using Equation 5, the time constant, which would be R*Ceap, would be 10.51191193 ms.

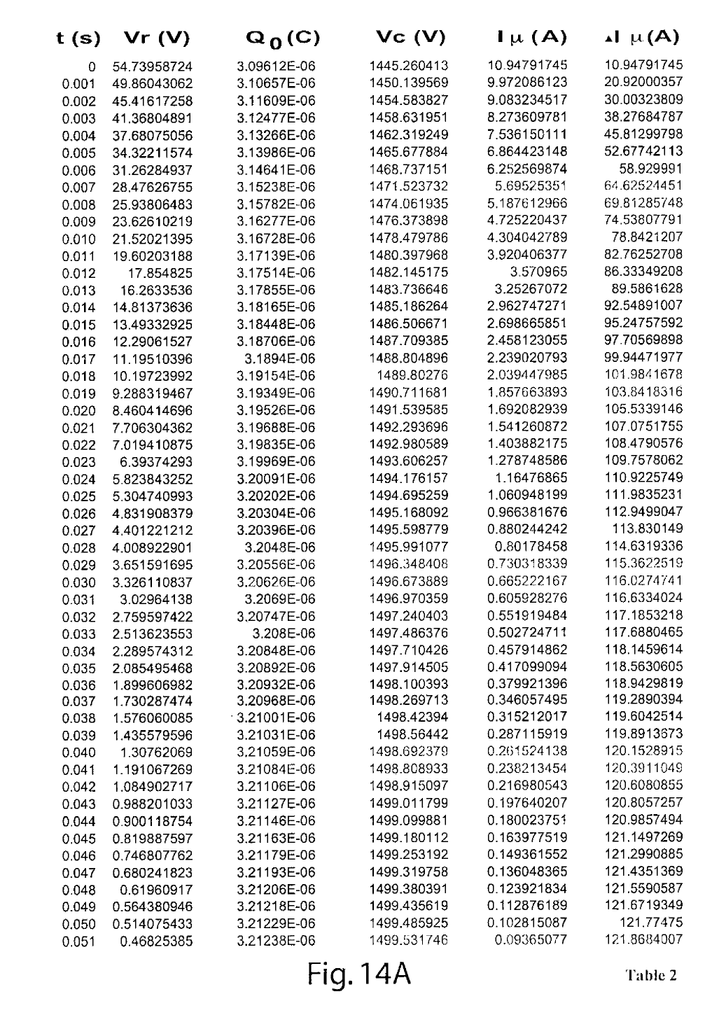

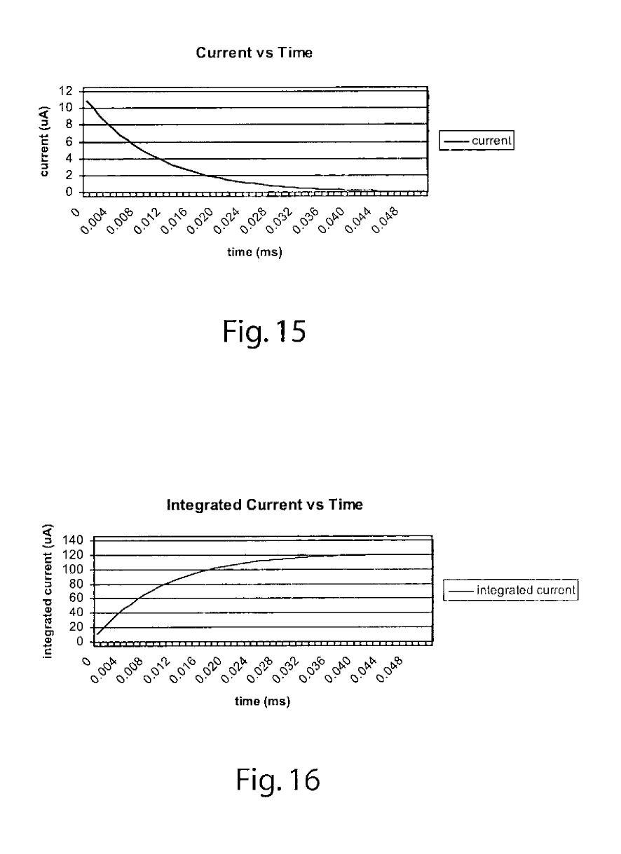

The change over time of the voltage Vc, in one ms increments, is shown in Table 1 of the drawings. Correspondingly, FIG. 13 illustrates the relationship of current versus time for the RC circuit 98. FIG. 14 illustrates the relationship between the integrated current and time for the circuit 98. It can be noted from Table 1 and FIGS. 13 and 14 that a change in height of 2 mm would change the capacitance Ceap to 2.142257725E 09 f. Correspondingly, the voltage across the capacitor Ceap, Vc, will drop to 1445.260413 V. Still further, FIGS. 15 and 16 show that the values of the current and integrated current over time for a 2 mm change in height are approximately twice the values for a 1 mm change in height. Accordingly, it would be possible to discriminate between a 1 mm displacement and a 2 mm displacement by sampling either current or voltage at 1000 Hz. This could occur even when sample times are offset by a full sample interval from the sample times which are illustrated.

The flexible element can be characterized as having a force constant K. The initial activation signal Va should be chosen so as to establish an active device neutral position and limit the maximum rate of change in position to 1 mm per 10 ms under application of the maximum expected external force. Notwithstanding the foregoing recommendation, it is clearly apparent that other values may be chosen without departing from the principal concepts of the invention.

The controller for use with the active support assembly can use the current, voltage and/or position signals for purposes of computing displacement. Given the value of the known activation signal Va, the magnitude of the externally applied forces can then be computed from the displacement. A force versus displacement curve of the flexible element can be assumed to be approximately linear. This curve can follow Hooke's Law, in accordance with Equation 9 as follows:

F=(Ks+Keapva)*X (Equation 9)

where Ks is the force constant of the flexible element and Keapva is the force constant of the electroactive polymer actuator for an activation signal, Va.

If the voltage or the current of the EAP actuator are sampled at equal intervals, the controller can specifically quantify the voltage or current change versus time. The initial position of the movable end of the EAP actuator can be characterized as x⁰. Using this initial position, the activation signal Va, and the current or voltage change versus time, the controller can search a lookup table for the corresponding position change of the movable end. This position change can be characterized as delta x. In this regard, the voltage or current change versus time for a set of incremental changes in the position of the movable end can be pre-recorded in the look up table for a set of initial positions, x⁰[i], and activation signals, Va[j]. Alternatively, the controller can also obtain position change from a position sensor. The change in position can then be used to calculate the external force (characterized as force F) applied to the active device, with the use of Hooke's Law in accordance with Equation 10 as follows:

F=(Ds+Keapva)*(x0+Δx) (Equation 10)

where x0 is the initial position and Δx is the position change of the movable end.

This same logic can be applied across all active devices within the array of EAP actuators. Accordingly, the controller can be utilized to generate a map of the forces which are applied across the entire active support surface. This can be accomplished by continuously sampling the currents which are drawn by the active devices. The controller can then update this “force map” or “pressure distribution map” in real time. This real time pressure distribution map can be used for several purposes. These purposes can include the following: (i) to focus pressure release methods in the areas of pressures; (ii) to determine position of the occupant; and (iii) to identify probable occupant posture and changes in probable occupant posture by continuously correlating the pressure distribution with a library of preloaded pressure distributions (where each preloaded distribution can be associated with a specific posture); (iv) to adapt the contours of the support surface for optimal postural support; (v) to translate virtual massage locations to physical massage locations; (vi) to record changes in probable occupant posture in a patient electronic medical record; and (vii) to generate a caregiver alert when probable occupant posture does not change over a pre-determined period of time.

For the foregoing purposes, it has been determined that pressure distribution can be updated at rates which are less than 1 Hz. Further, instead of sampling the actuator current or voltage change, it would also be possible for the controller to periodically measure the capacitance of the EAP actuator by injecting what can be characterized as an alternating current, I(t), and measuring the voltage across the actuator, Vc(t) (or across the series resistor, Vr(t)). In this regard, and in accordance with Equation 11 below, the controller can use the current, the measured voltage and the resistor value, R, to compute the impedance, Zc, of the EAP actuator at a frequency, w.

Vc(t)=1(t)*(Zc+R)

Zc=Vc(t)I(t)−R

1(t)=Ipeak*sin(wt) (Equation 11)

where Zc is the impedance of the electroactive polymer actuator at the frequency w.

The controller can utilize this impedance to essentially find the corresponding position of the movable end by means of a lookup table. That is, the impedance verses position of the movable end can be prerecorded. Based on the position and the activation signal level Va, the controller can then compute the magnitude of the externally applied forces.

For purposes of mimicking of a passive surface, the controller would be required to update the pressure distribution measurement at rates approaching 1000 Hz. The time constants of the EAP actuators must therefore be small. This is required so that the controller can obtain accurate measurements of the current or voltage changes. It is also required so that the controller can derive displacements of the corresponding support plates, compute magnitudes for the externally applied forces and respond with activation signals before the displacements of the support plates become noticeable to the occupant. It is reasonable to expect that displacements exceeding 1 mm would be noticeable to certain occupants. If a maximum acceleration is assumed of the support plate of 9.8 m/s², the support plate can obtain a displacement of 1 mm after 10 ms, assuming a starting velocity of zero. A time constant between 5 ms and 50 ms would allow the controller to accurately determine the current or voltage change within 10 ms, and would therefore dictate a sample rate between 200 Hz and 1000 Hz.

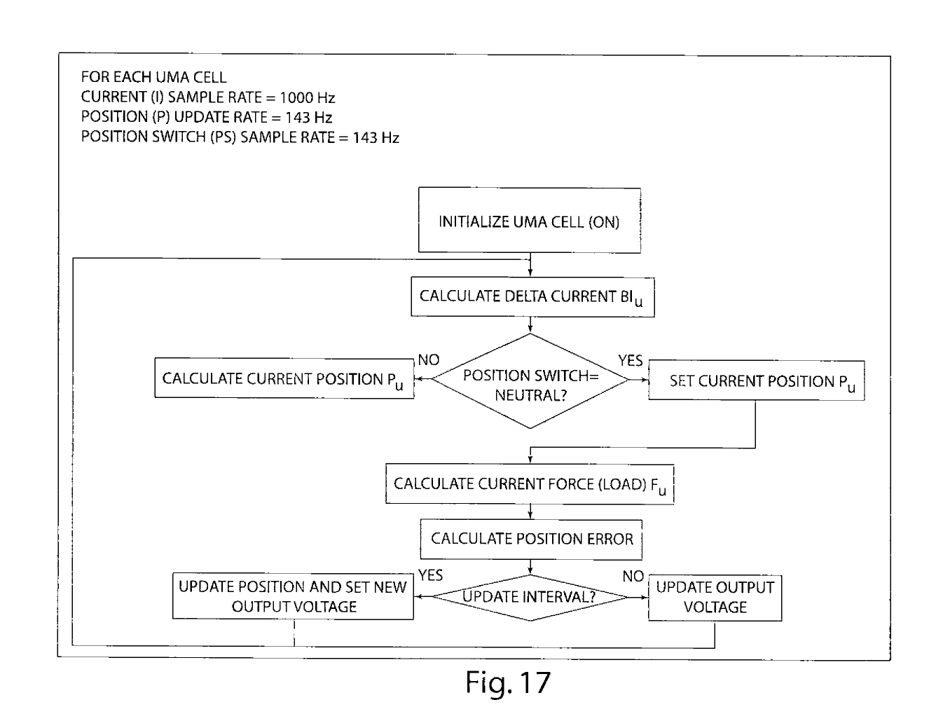

With respect to the foregoing, FIGS. 17 and 18 illustrate one embodiment of an active support assembly which is capable of mimicking a passive surface. The drawings illustrate a sequence diagram for a software process, along with a block diagram for the associated electronics.

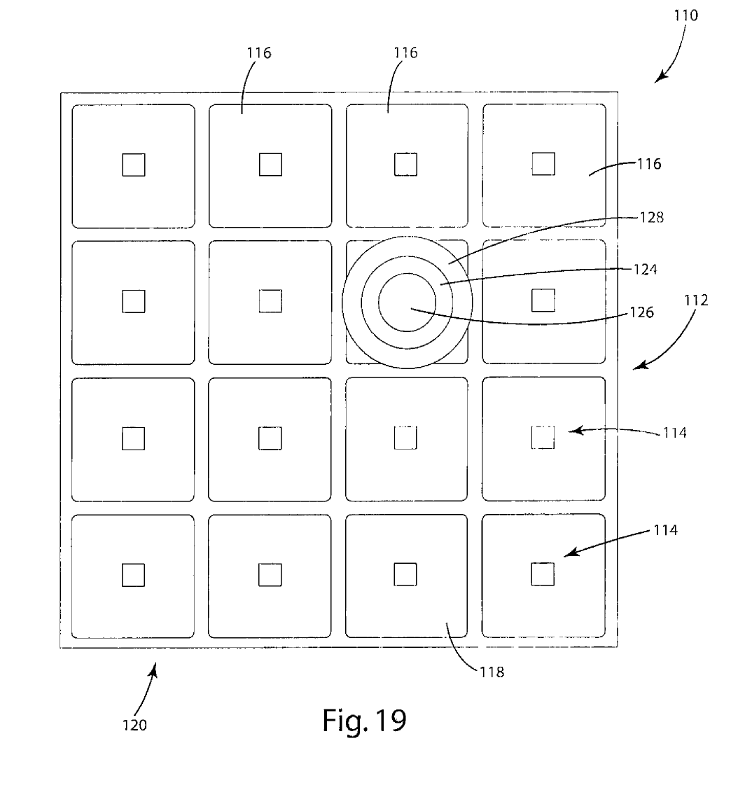

For purposes of further explanation of embodiments in accordance with the invention, a surface characterization assembly 110 is illustrated in FIG. 19. As shown therein, the characterization assembly 110 comprises an array 112 of a series of nodes 114. Each node 114 can be characterized as consisting of a rigid disk 116. Each of the nodes 114 corresponds to an active device 118 within what can be characterized as an active support assembly 120 as illustrated in FIG. 19. Each of the nodes 114 in the surface characterization assembly 110 can further be described as being aligned with and having the same diameter as the corresponding active device 118. Further, each of the nodes 114 includes an accelerometer 122. With this configuration, and with respect to the accelerometers 122, it should be noted that the “tension” which will exist between adjacent nodes 114 of the surface characterization assembly 110 will be relatively negligible.

The surface characterization assembly 110 as shown diagrammatically in FIG. 19, can be applied to a passive surface, such as a waterbed or the like. The surface characterization assembly 110 can be utilized to cover the entire area which is to be “characterized.” Each of the nodes 114 can be bonded securely to the passive surface so as to create an array 112 of what may be characterized as “virtual” devices. For purposes of characterization, a known force of relatively high amplitude and short duration (essentially in the form of an “impulse”) can be applied to each of the nodes or virtual devices 114. Each node in the array can be characterized as a node N[i][j]. The impulses can be applied in sequence, using a device such as a hammer 124 as shown in FIG. 19. The hammer 124 can include a ring-shaped head 126. A hole 128 of the ring-shaped head 126 can be aligned with the accelerometer 122 of the then current node N[i][j] being characterized. This alignment will prevent shattering or other damage to the accelerometer 122. For each hammer strike or impulse, the output of the accelerometer 122 associated with the corresponding node can be characterized as A[n][m], where the small n and small m signify the particular accelerometer 122 and corresponding node N[i][j]. This output can be sampled, for example, at a rate of 200 Hz to 1000 Hz. The sampling can continue to occur until the acceleration of the particular node 114 decreases to less than 1 to 10 cm/s per ms, or, alternatively, the displacement decreases to less than 1 to 10 nm. The resultant data set for each node N[n][m] can be characterized as representing the impulse response of each corresponding virtual device in the passive surface to a force applied to a particular node N[i][j]. It should be noted that the data set for each node may actually be decimated by low-pass filtering and down-sampling.

Following the characterization of all of the nodes, the individual impulse responses of all points on the passive surface to a force applied to any specific point on the surface can be known with a precision which is essentially determined by the relative diameter of each of the nodes 114.

General concepts associated with calibration of any of the active support assemblies in accordance with the invention will now be described. Specifically, each of the active support assemblies can be calibrated by applying a range of discrete forces to each constituent active device across a range of activation signal levels (measured in volts) and positions. When the discrete forces are applied, sampling can occur of both the position change of an output shaft associated with each EAP actuator, along with the current flow through each EAP actuator. Preferably, the sampling will occur at a rate of between 200 Hz to 1000 Hz. The position change may be sensed using various instrumentation. For example, one device which may be utilized is commonly referred to as a “linear variable differential transformer” or “LVDT.” An example of such a differential transformer is manufactured and marketed by Schaevitz Sensors and is referred to as a “Sensor LVDT DC-SE SERIES100MM.” The part number is 02560995-000 and the Digi-Key part number is 356-1026-ND. Correspondingly, current may be sampled through the use of a Hall effect sensor. One type of such sensor is manufactured by Analog Devices, and is identified as manufacturer part number ADXL210AE. The Digi-Key part number is ADXL210AE-ND. For each of the active devices, the resulting data set will describe a curve representing displacement versus forces at different voltages and positions for different applied forces. As with the surface characterization, the data set for each active device may be decimated by use of low-pass filtering and down-sampling.

For purposes of completing in full a detailed explanation of an active surface assembly embodiment in accordance with the invention, utilizing push-pull EAP actuators, information can currently be obtained from Artificial Muscle, Inc. or “AMI.” AMI manufactures what are characterized as “Universal Muscle Actuators” or “UMAs.” These actuators are push-pull actuators which are based on EAP technology.

With the use of push-pull actuators from AMI, input voltages are recommended within the range of 1 to 24 VDC from conventional batteries. However, the actuators can also be designed so as to work with 100-240 VAC 50-60 Hz input. The average power which is typically drawn for these actuators is relatively low. Also, since the actuators are essentially a capacitive load, power draw will primarily occur when the device is charging. The actual power required will be dependent upon the capacitance of the device.

The AMI actuators can operate over a relatively broad range of frequencies. Certain actuators are designed to run at less than 1 Hz so as to maximize displacement. Correspondingly, other actuators are designed to run as high as 17 Khz. In this regard, it can be seen that frequency is one of the controllable parameters which can be utilized to optimize actuator performance.

With respect to control of the actuator devices, the AMI actuators typically include integration of appropriate electronics for purposes of driving each actuator. With respect to operating strains of the actuators, typical operating strains (where reliability is maintained) in the no-load state will be in the range of 5 to 15% over the active length of the device. Maximum strains far exceeding this range have been physically realized. However, there is clearly an operational trade-off between strain and life cycles, when devices are loaded. For applications which require relatively high lifetimes, the actuators are required to be designed so as to operate at strain levels below 15 percent. It should also be noted, however, that the strain is also dependent upon frequency. That is, as frequency is increased, the strain will decrease. The strain frequency response will be dependent upon material properties, configuration design and control electronics.

With respect to forces which can be exerted by the actuators, there are limits in a physically realized given volume of an actuator in any given component size. In this regard, a linear relationship can be assumed between force and the number of layers of the support assemblies. For example, a one-layer set of devices can have a force of 0.5 Newtons. Correspondingly, 20-layer devices of the configuration can be shown to have a blocked force of 10 Newtons. From the foregoing description, it should also be somewhat apparent that the displacement will be a “trade-off” with force. That is, an actuator can start with a maximum force level (i.e. blocking force), and then decrease as it expands outward with voltage, until such time as a zero force is realized at maximum displacement. As the number of layers of devices increases, the forces will increase as well. However, the devices will, correspondingly, become physically larger.

The actuators from AMI can operate over a relatively wide range of temperatures. However, temperature can clearly affect performance of the actuators. Also, different dielectric materials operate at differing operating temperature ranges. For example, an advantage of a silicone-based dielectric is that the same can operate well below freezing. Dielectrics will tend to improve in performance at relatively higher temperatures (i.e. exceeding 50 C) as a result of the relative decrease in viscoelasticity.

Various other statements can also be made with respect to the AMI actuators. The “power to weight” ratios will vary by configuration. Also, in certain applications and configurations, AMI actuators may exhibit some hysteresis. Regarding specific performance configurations, certain data has been obtained through physically realized and measured systems. For example, it can be assumed that a subject has an average weight of 171.18 pounds. The subject is seated on a chair having a surface area of 16×16 inches. The chair is topped with an air-filled overlay and produces, on average, a total contact area of approximately 220 in², an average pressure of 0.93 psi and a peak pressure of 3.19 psi. Correspondingly, subjects lying on a mattress, where the weight is distributed over a larger area, would be expected to produce lower average and peak pressure values. Twenty-layer devices of a configuration have demonstrated a blocked force (at 0 inches displacement) of ten Newtons or 2.25 pounds. Assuming that an actuator device can be fitted with a 1×1 inch support plate and biased with a flexible element providing one pound of additional resistive force (thus yielding a total blocking force of 3.25 pounds), an array of such active devices could be capable of supporting and possibly even lifting the subjects described having the foregoing average weight. Further, individual devices should be capable of retracting their support places so as to reduce pressure on a relatively precise (e.g. 1×1 inch) basis by compressing their associated flexible elements. The depth of the compression would be determined by the three elements of Hooke's Law: (i) the total applied force (occupant load+active device force); (ii) the position of the flexible element; and (iii) the force constant of the flexible element.

With the foregoing in mind, a surface area can be assumed of nine square feet having 1×1 inch support plates. This configuration will provide 1296 active devices. If the current/voltage across one layer of each device was sampled at 1000 Hz, the sample would be 772 nsecs. A microprocessor which runs at 200 Mhz has a cycle time (i.e. clock interval) of nsecs. Accordingly, 154 cycle times would be available to process each sample. If activation signals were computed for every ten samples, 1540 cycle times would be available for each of these computations. Correspondingly, doubling the microprocessor clock speed would double the number of cycles available for processing sample data and computing activation signals. Also, reducing the current/voltage sample rate from 1000 Hz to 200 Hz would essentially “quintuple” the number of available cycles.

The following has described several embodiments of active support surfaces or active support assemblies in accordance with the invention. It will be apparent to those skilled in the pertinent arts that other embodiments of the invention can be designed. That is, the principles of the invention are not limited to the specific embodiments of active support assemblies as described herein. Accordingly, it will be apparent to those skilled in the pertinent arts that modifications and other variations of the above-described illustrative embodiments of the invention may be effected without departing from the spirit and scope of the novel concepts of the invention.

1. An active support assembly adapted for use with mattresses, overlays and seating, said active support assembly comprising:
a frame assembly;
a plurality of support plates;
an array of active devices, wherein said active devices comprise stationary ends fixedly attached to said frame assembly, and movable ends operatively attached to said support plates, said active devices further comprising:
electroactive polymer actuators, wherein a first section of each electroactive polymer actuator of a subset of said electroactive polymer actuators is attached to said stationary end of said active device, and a second section of each of said electroactive polymer actuators of said subset of said electroactive polymer actuators is attached to said movable end of said active device;
each of said active devices functions as a sensor, wherein movement of said movable end of each of said active devices resulting from the application of an external force to said support plate attached to said active device effects a change in an attribute of said electroactive polymer actuator, and wherein said change in said attribute is measurable;
each of said active devices also functions as an actuator, wherein application of an activation signal to said electroactive polymer actuator effects a change in a further attribute of said electroactive polymer actuator, and wherein said change in said further attribute forcibly moves said movable end of said active device and said attached support plate;
a controller in operative communication with each of said electroactive polymer actuators of each of said active devices, wherein said controller continuously measures said change in said attribute of said electroactive polymer actuator due to movement of said movable end of said active device and said attached support plate;
said controller uses said measured change in said attribute so as to compute a change in acceleration, velocity and position of said movable end and in the magnitude of said applied force, and wherein said controller uses the change in acceleration, velocity and position of said movable end and the magnitude of said applied force to selectively apply an activation signal to said electroactive polymer actuator and effect a change in said attribute of said electroactive polymer actuator, and wherein said change in said attribute results in a desired change in said acceleration, velocity and position of said movable end of said active device, and the attached support plate and in the magnitude of said resistive force; and
said support plates form a plane of support under the application of certain of said activation signals and certain of said external forces.

2. An active support assembly in accordance with claim 1, characterized in that said frame assembly comprises:
a rigid or semi-rigid layer;
a compliant layer, wherein said compliant layer is configured to comply with larger, slower forces applied externally and resist smaller, faster forces generated internally by said active devices under application of said activation signals; and
wherein said external forces are mechanically coupled to said frame assembly through linkage of said support plates to said active devices and to said frame assembly.

3. An active support assembly in accordance with claim 1, characterized in that said array of active devices is configured as a plurality of sub-array layers, each of said sub-array layers comprising:
a subframe fixedly attached to said frame assembly;
a plurality of extension rods having first ends and second ends;
said active devices of each sub-array layer are offset at least one radii from the active devices of all succeeding sub-array layers;
said active devices having stationary ends fixedly attached to said subframe and movable ends operatively attached to said first ends of said extension rods;
said extension rods penetrating all succeeding sub-array layers through interstices of said sub-array layer active devices;
said second ends of said extension rods being operatively attached to said support plates;
lengths of said extension rods being configured so as to dispose said support plates on a plane under application of certain activation signals and certain external forces; and
said plane combining with planes of succeeding sub-array layers so as to form a single plane of support.

4. An active support assembly in accordance with claim 1, characterized in that:
said frame assembly comprises a plurality of modules, each of said modules being characterized as a modular frame assembly; and
each of said modular frame assemblies is rigidly or pivotally attached to others of said modular frame assemblies so as to form a multi-frame assembly.

5. An active support assembly in accordance with claim 4, characterized in that:
a compliant material is fixedly inserted between adjacent ones of said modular assemblies which are attached to each other;
said compliant material compresses when faces of said modular frame assemblies are parallel, and expands so as to fill a space created when said faces of said modular frame assemblies are not parallel; and
said expanded material forms a continuous support surface between said adjacent ones of said attached modular frame assemblies.

6. An active support assembly in accordance with claim 1, characterized in that:
said support plates are pivotally mounted to said movable ends of said active devices;
said support plates pivot in response to application of a non-uniform external force; and
said support plates are pre-loaded so as to assume positions orthogonal to lengthwise directions of active devices to which said support plates are mounted in the absence of external forces.

7. An active support assembly in accordance with claim 6, characterized in that said pre-load of said support plates consists of one or a combination of at least two of the following means for pre-loading: spring; air-filled cell; gel-filled cell; fluid-filled cell; foam cell.

8. An active support assembly in accordance with claim 1, characterized in that each of said support plates comprises a layer of compliant material.

9. An active support assembly in accordance with claim 1, characterized in that said layer of compliant material covers a layer of rigid or semi-rigid material.

10. An active support assembly in accordance with claim 1, characterized in that said support plates are joined together by compliant connector ribbon so as to form a network of support plates.

11. An active support assembly in accordance with claim 10, characterized in that:
said support plates and said connector ribbon comprise a single sheet of the same material; and
the material thickness of said single sheet of the same material is configured so as to effect different levels of compliance for said support plates and connector ribbon.

12. An active support assembly in accordance with claim 10, characterized in that:
said support plates and said connector ribbon comprise a single sheet of the same material; and
the size and shape of perforations in said single sheet of the same material are configured so as to effect different levels of compliance for said support plates and said connector ribbon.

13. An active support assembly in accordance with claim 1, characterized in that:
each of said active devices comprises a resistor in series with a corresponding one of said electroactive polymer actuators;
each of said corresponding electroactive polymer actuators forms a flexible capacitor, so that each of said resistors and each of said corresponding electroactive polymer actuators forms a resistor-capacitor circuit; and
each of said resistors is configured so as to establish a time constant of said corresponding resistor-capacitor circuit.

14. An active support assembly in accordance with claim 1, characterized in that:
each of said active devices comprises at least one sensor so as to measure said change in said attribute of said corresponding electroactive polymer actuator; and
each of said sensors consists of one of the following elements, or a combination of at least two of said following elements: capacitance sensor; current sensor; voltage sensor; position sensor; accelerometer.

15. An active support assembly in accordance with claim 1, characterized in that:
said assembly comprises a stacking or bonding of multiple layers of said electroactive polymer actuators so as to provide a set of multi-layer electroactive polymer actuators; and
as a result of said stacking or bonding, said actuators are capable of exerting relatively greater forces than a single layer electroactive polymer actuator under the application of an activation signal.

16. An active support assembly in accordance with claim 1, characterized in that each of said active devices comprises:
a flexible element, said flexible element being an energy storage and return element having a movable end and a stationary end fixedly attached to said frame assembly;
an output cap;
a mask comprising a section of material having an opening so as to receive said flexible element;
said electroactive polymer actuators, lay between said flexible element and said mask;
said mask being positioned in a manner so that it is lowered over said flexible element and bonded to said frame assembly, thus causing said electroactive polymer actuator to be stretched over said flexible element;
said output cap is operatively attached to said movable end of said flexible element, so that a non-active portion of said electroactive polymer actuator lies between said movable end of said flexible element and said output cap;
said electroactive polymer actuator further being configured so as to forcibly retract said movable end and said output cap, thus compressing said flexible element, or, alternatively, said electroactive polymer actuator forcibly extending said movable end and said output cap, thus expanding said flexible element, under application of selected activation signals; and
said output cap is operatively attached to said support plate.

17. An active support assembly in accordance with claim 16, characterized in that each of said flexible elements consist of one or more of the following elements, or a combination of at least two of said following elements: spring; air-filled cell; gel-filled cell;
fluid-filled cell; foam cell.

18. An active support assembly in accordance with claim 16, characterized in that each of said active devices is configured as an active device section, and each of said active device sections comprises:
a flexible element section;
a mask section;
an electroactive polymer actuator section;
a plurality of output caps;
said flexible element section comprises an array of flexible elements, with said flexible elements each having a stationary end fixedly attached to said frame assembly and said movable ends;
said mask section comprising a single section of material having a plurality of openings disposed so as to receive said flexible elements in said flexible elements section;
said electroactive polymer actuator section comprises a single section of material having a plurality of electroactive polymer actuators, said electroactive polymer actuators being aligned with said flexible elements in said flexible element section;
each of said electroactive polymer actuator sections is configured so as to lie between said flexible element section and said mask section;
said mask section is lowered over said flexible element section and bonded to said frame assembly, thus causing said electroactive polymer actuators to be stretched over the flexible elements in said flexible element section;
said output caps being operatively attached to said movable ends of said flexible elements, so that a non-active portion of each of said electroactive polymer actuator sections lies between said movable ends of said flexible elements and said output caps;
said electroactive polymer actuators being configured so as to forcibly retract said movable ends and said output caps, thus compressing said flexible elements, or, alternatively, said electroactive polymer actuators forcibly extending said movable ends and said output caps, thus expanding said flexible elements, under application of selected activation signals; and
said output caps are operatively attached to said support plates.

19. An active support assembly in accordance with claim 1, characterized in that each of said active devices comprises an electroactive polymer push-pull actuator, with each of said electroactive polymer push-pull actuators comprising:
an output shaft;
an output disk;
first and second outer frames;
first and second electroactive polymer actuators, wherein said first electroactive polymer actuator is suspended between said first outer frame and said output disk, and said second electroactive polymer actuator is suspended between said second outer frame and said output disk, and said second outer frame is parallel to said first outer frame and offset from said first outer frame by a spacer;
said first outer frame and/or said second outer frame are configured as said stationary end of said active device, with said stationary end being fixedly attached to said frame assembly;
said first electroactive polymer actuator exerting a pulling force on said output disk in one direction or, alternatively, said first electroactive polymer actuator exerts a pushing force on said output disk in an opposite direction under application of selected activation signals;
said second electroactive polymer actuator exerting a pulling force on said output disk in one direction or, alternatively, said second electroactive polymer actuator exerts a pushing force on said output disk in an opposite direction under application of selected activation signals;
directions and magnitudes of said forces exerted by said first electroactive polymer actuator and said second electroactive polymer actuator on said output disk are configured to as forcibly move said output disk;
said output shaft having a first end operatively attached to said output disk and a second end configured as said movable end of said active device; and
said movable end is operatively attached to said support plate.

20. An active support assembly in accordance with claim 19, characterized in that:
each of said electroactive polymer push-pull actuators is biased with a flexible element;
said flexible element is an energy storage and return element, and is optionally compressed;
said flexible element having a stationary end connected to one of the following elements, or a combination of at least two of the following elements: frame assembly; subframe; first outer frame; second outer frame;
said flexible element having a movable end operatively attached to said output disk of said electroactive polymer push-pull actuator.

21. An active support assembly in accordance with claim 19, characterized in that:
each of said active devices comprises a stack of at least two electroactive polymer push-pull actuators; and
each of said additional actuators are configured so as to increase displacement produced by said active device.

22. An active support assembly in accordance with claim 1, characterized in that each of said active devices comprises an electroactive polymer roll actuator, each of said electroactive polymer roll actuators comprising:
two electroactive polymer actuators;
a mounting cap;
an output cap;
a flexible element, comprising an energy storage and return element, with said flexible element being in a compressed state or a non-compressed state;
said two actuator polymer actuators are wrapped around said flexible element so as to form a cylinder having a first end and a second end, with said first end configured as said stationary end of said active device and said second end is configured as said movable end of said active device;
said mounting cap is attached to said stationary end and fixedly attached to said frame assembly;
said output cap is attached to said movable end and operatively attached to said support plate;
said two electroactive polymer actuators are configured so as to forcibly retract said movable end and said output cap, thus compressing said flexible element, or, alternatively, said two electroactive polymer actuators forcibly extend said movable end and said output cap, thus expanding said flexible element, under application of selected activation signals.

23. An active support assembly in accordance with claim 1, characterized in that:
said controller comprises a plurality of controller means for selectively applying activation signals to said active devices in response to changes in the accelerations, velocities and positions of said movable ends and in magnitudes of said external forces; and
said controller further comprises means operable by an occupant/user with the capability of selecting separate ones of controller means to be used for selectively applying said activation signals.

24. An active support assembly in accordance with claim 23, characterized in that said controller means comprise one or more of the following means:
means for sensing and relieving sustained compression of tissues occurring in occupants of said mattresses, overlays and/or seating;
means for mimicking a response of a passive support surface to externally applied forces;
means for generating surface vibration so as to reduce a coefficient of friction of said support surface;
means for sensing and reducing external vibrational forces communicated to said occupant;
means for determining a location of said occupant on said support surface;
means for identifying a probable posture of said occupant and recording changes in said probable posture within a patient electronic medical record;
means for generating an alert in the event that said probable occupant posture does not change for a predetermined interval of time;
means for facilitating occupant/user-directed massage;
means for adapting support surface contours in response to changes in said occupant location and said probable occupant posture;
means for controlling firmness and stability of said support surface; and
means for synthesizing one or more responses by said support surface to said externally applied forces;

25. An active support assembly in accordance with claim 23, characterized in that said support surface is divided into a plurality of zones, and each of said zones may be acted upon by differing ones of said controller means in a simultaneous manner.

26. A method for sensing and relieving sustained compression of tissues occurring in occupants of mattresses, overlays and/or seating, said method using an array of active devices having stationary ends fixedly attached to a frame, and movable ends configured so as to form a support surface, said method comprising:
detecting movements of said movable ends and outputting sensor signals associated with said detected movements, said detection being based on deformation of electroactive polymer actuators;
reading said sensor signals;
computing magnitudes of external forces associated with movements of said movable ends, and identifying the locations of peak forces, based on said reading of said sensor signals;
selectively applying activation signals to said active devices, wherein said activation signals cause said active devices to forcibly move their movable ends, said movements being generated by deformation of said electroactive polymer actuators;
causing said active devices at and proximal to said locations of said peak forces to retract their movable ends by variable amounts, so as to reduce resistive forces on occupant surface tissues at and proximal to locations of said peak forces;
optionally causing said active devices distal to said locations of said peak forces to extend their movable ends by variable amounts so as to increase said resistive forces on said occupant surface tissues at said locations distal to said peak forces;
configuring velocities of said movable ends so as to limit impact forces on said occupant, until said locations of said peak forces change or a predetermined set time interval has elapsed, said time interval being capable of being set to ranges of seconds to minutes;
selectively updating said activation signals, so that said updated activation signals cause said active devices having movable ends in a retracted state to extend said movable ends by variable amounts so as to increase said resistive forces on said occupant surface tissues at corresponding locations;
configuring said velocities of said movable ends so as to limit said impact forces on said occupant, wherein said updated activation signals optionally cause said active devices having movable ends in extended state to retract said movable ends by variable amounts so as to reduce said resistive forces on said occupant surface tissues at corresponding locations; and
effecting changes in magnitudes and directions of stress vectors in occupant deep tissues, through said changes in said resistive forces on said occupant surface tissues and interrupting sustained compression of said deep tissues.

27. A method of mimicking a response of a passive support surface to externally applied forces using an array of active devices having stationary ends fixedly attached to a frame, and movable ends configured so as to form a support surface, said method comprising:
detecting movements of said movable ends and outputting sensor signals associated with said detected movements, and with said detection being based on deformation of electroactive polymer actuators;
reading said sensor signals;
computing magnitudes of external forces associated with said movements of said movable ends;
selectively applying activation signals to active devices, with said activation signals causing said active devices to forcibly move said movable ends by variable amounts, said movement being generated by deformation of electroactive polymer actuators;
computing direction and velocity of said forcible movement of each of said active devices using principles of superposition, as applied to an aggregate of direct and indirect impulse responses of said active device, said direct impulse response defined by a position-versus-time curve of said movable end of said active device in response to an external force applied to said active device, and said indirect impulse response defined by a position-versus-time curve of said movable end of said active device in response to an external force applied to an adjacent one of said active devices;
said direct impulse response of said active device being based on a measured response of said passive support surface to a high amplitude, short duration force applied at a location coincident with a location of said active device in said array of active devices;
said indirect impulse responses of said active device being based on measured responses of said passive support surface to a series of high amplitude, short duration forces sequentially applied at locations coincident with locations of adjacent active devices in said array of active devices; and
said high amplitude, short duration forces approximate an impulse function.

28. A method in accordance with claim 27, characterized in that said method further comprises:
adjusting firmness and stability of said mattresses, overlays and/or seating;
selecting a desired level of firmness and stability;
decreasing amplitude of said direct impulse responses in response to an increase in firmness;
increasing said amplitude of said direct impulse responses in response to a decrease in firmness;
decreasing amplitude and/or length and/or oscillations of said indirect impulse responses in response to an increase in stability; and
increasing said amplitude and/or length and/or oscillations of said indirect impulse responses in response to a decrease in stability.

29. A method in accordance with claim 27, said method further comprising:
synthesizing said response of said mattresses, overlays and/or seating to said externally, applied forces;
defining amplitudes, frequencies, and damping of oscillations of said direct impulse responses; and
defining amplitudes, frequencies, and damping of oscillations of said indirect impulse responses.

30. A method for sensing and reducing external vibrational forces communicated to occupants of seating using an array of active devices having stationary ends fixedly attached to a frame and movable ends configured so as to form a support surface, said method comprising:
detecting movements of said movable ends and outputting sensor signals associated with said detected movements, said detection based on deformation of electroactive polymer actuators;
reading said sensor signals;
on the basis of said sensor signals, identifying components of said detected movements that are periodic, and measuring periods, amplitudes and phases of said periodic movements of said movable ends;
computing oppositional movements, wherein each of said oppositional movements is configured so as to have the same period and amplitude as one of said measured periodic movements and a phase difference of 180 degrees with said measured periodic movement;
selectively applying activation signals to said active devices, said activation signals causing said active devices to forcibly move their movable ends, said movement being generated by deformation of said electroactive polymer actuators; and
using principles of superposition to compute said period, amplitude and phase of said forcible movement of each of said active devices, wherein said principles of superposition are applied to an aggregate of said oppositional movements computed for said active device.

31. A method of generating surface vibration in mattresses, overlays and/or seating so as to reduce a coefficient of friction of a support surface using an array of active devices having stationary ends fixedly attached to a frame and movable ends configured so as to form said support surface, said method comprising:
applying activation signals to said active devices, wherein said activation signals cause said active devices to forcibly move said movable ends, said movement being generated by deformation of electroactive polymer actuators, and where said movable ends are extended and retracted at frequencies of at least 10 Hz;
for specific intervals of time, retracting said movable ends of one group of said active devices and extending said movable ends of a second group of said active devices; and
reducing an area of surface contact between an occupant and said mattresses, overlays and/or seating.

32. A method for determining the location of occupants of mattresses, overlays and/or seating using an array of active devices having stationary ends fixedly attached to a frame and movable ends configured so as to form a support surface, said method comprising:
detecting movements of said movable ends and outputting sensor signals associated with said detected movements, said detection being based on deformation of electroactive polymer actuator means;
reading said sensor signals;
based on said sensor signals, computing pressure distribution across said support surface and computing said location of said occupant based on said pressure distribution.

33. A method in accordance with claim 32, characterized in that said method further comprises processes for facilitating occupant/user-directed massage, with said method further comprising the steps of:
selecting a massage tool icon from a plurality of massage tool icons presented on a pressure-sensitive graphical display;
dragging said massage tool icon across an image of a body presented on said pressure-sensitive graphical display;
selecting a location for massage and adjusting forces applied to said pressure-sensitive graphical display to select an intensity for said massage;
using said computed location of said occupant to identify said active devices corresponding to said selected massage location;
selectively applying activation signals, to said corresponding active devices, wherein said activation signal levels are based on said selected massage intensity; and
through said activation signals causing said active devices to forcibly move said movable ends, said movement being generated by deformation of said electroactive polymer actuators.

34. A method in accordance with claim 32, characterized in that the method further comprises processes for identifying a probable posture of said occupant based on the computed pressure distribution, said method further comprising the steps of:
correlating said computed pressure distribution against a plurality of pre-loaded pressure distributions, wherein said pre-loaded pressure distributions correspond to a plurality of unique occupant postures; and
selecting said probable occupant posture based on values of correlation coefficients determined from said correlation of said computed pressure distribution.

35. A method in accordance with claim 34, characterized in that said method further comprises the step of recording a change in said probable occupant posture in an electronic medical record.

36. A method in accordance with claim 34, characterized in that said method further comprises the step of generating an alert in the event said probable occupant posture does not change for a predetermined interval of time.

37. A method in accordance with claim 34, characterized in that said method further comprises processes for adapting contours of said support surface based on changes in said occupant location and said probable posture, said method further comprising the steps of:
selectively applying activation signals to said active devices, wherein said activation signals cause said active devices to forcibly move said movable ends;
said movement of said movable ends being generated by deformation of said electroactive polymer actuator means; and
for certain ones of said occupant locations and said postures, retracting or extending said active devices located at said certain locations.

38. A method in accordance with claim 37, characterized in that said method further comprises extending ones of said active devices located in a thoracic back region of a supine occupant, so as improve respiration of said occupant.

39. A method in accordance with claim 37, characterized in that said method further comprises extending ones of said active devices located in a popliteal region of a supine occupant, so as to reduce lower back strain of said occupant.

40. A method in accordance with claim 37, characterized in that said method further comprises extending ones of said active devices located forward of ischial tuberosities of a reclining occupant, so as to prevent sliding of said occupant.

41. A method in accordance with claim 37, characterized in that said method further comprises extending ones of said active devices located lateral to a thorax of a seated occupant, so as to provide lateral support of said occupant.

42. A system for relieving sustained compression of tissues occurring in occupants of mattresses, overlays and/or seating, including isolating vibrational forces acting on and/or enhancing comfort and/or postural support afforded said occupants, said system comprising:
a frame;
a plurality of active material based devices having stationary ends fixedly attached to said frame and movable ends configured so as to form a support surface, and wherein movement of any one of said movable ends due to application of an external force effects a change in an attribute of an active material, said change in said attribute being measurable;
means for effecting a change in said attribute of said active material through application of an activation signal to said active material, said change in said attribute forcibly moves said movable end;
controller means are in operative communication with said active material of said active material based devices, said controller means continuously measuring said changes in said attributes of said active material due to movement of said movable ends;
said controller means using said measured changes in said attributes so as to compute changes in positions, velocities and accelerations of said movable ends, and in magnitudes of said external forces;
controller means uses said changes in said positions, velocities and accelerations of said movable ends and in said magnitudes of said applied forces to selectively apply said activation signals to said active material and effect changes in said attributes of said active material; and
said changes in said attributes cause desired changes in said accelerations, velocities and positions of said movable ends, and in magnitudes of resistive forces.

43. A system for relieving sustained compression of tissues occurring in occupants of mattresses, overlays and/or seating, and isolating vibrational forces acting on, and/or enhancing the comfort and/or postural support afforded occupants of said mattresses, overlays and seating, said system comprising:
a frame;
a plurality of active devices, wherein said active devices comprise actuators, sensors, stationary ends and movable ends;
said stationary ends being fixedly attached to said frame and said movable ends being configured so as to form a support surface;
controller means in operative communication with said sensors and said actuators, said controller means continuously sampling information provided by said sensors so as to compute changes in positions, velocities and accelerations of said movable ends and in magnitudes of externally applied forces;
said controller means comprises means responsive to said changes in said positions, velocities and accelerations of said movable ends and in the magnitudes of said externally applied forces so as to selectively apply activation signals to said actuators; and
wherein said activation signals cause said actuators to effect desired changes in said positions, velocities and accelerations of said movable ends and in magnitudes of resistive forces.

44. A system for enhancing comfort and/or postural support afforded occupants of mattresses, overlays and/or seating, said system comprising:
a plurality of active devices, each of said active devices incorporating an active material, and said plurality of active devices configured so as to form a support surface;
controller means in operable communication with said active devices, wherein said controller means comprises means for selectively applying activation signals to said active material of said active devices and effecting changes in an attribute of said active material; and
said changes in said attribute result in desired changes in movement and resistive forces of said active devices, and in the shape and behavior of said support surface.

45. A system in accordance with claim 44, characterized in that:
said system further comprises means for measuring changes in said attribute of said active materials of said active devices due to externally applied forces; and
said system further comprises means for providing said measurements of said changes in said attribute of said active material to said controller means for selectively applying said activation signals to said active material of said active devices.

46. A system for relieving sustained compression of tissues occurring in occupants of mattresses, overlays and/or seating, and isolating vibrational forces acting on, and/or enhancing comfort and/or postural support afforded said occupants, said system comprising:
an array of active support elements forming a plane of support, wherein each of said active support element is operable so as to move independently of other ones of said active support elements;
means for initializing positions and velocities of said active support elements magnitudes of resistive forces of said active support elements;
means for continuously sensing movements of said active support elements resulting from application of external forces;
means for computing magnitudes of said applied external forces based on sensed movements;
means for selectively applying said activation signals to said active support elements, based on computed changes in magnitudes of said applied external forces; and
wherein said activation signals effect changes in positions and velocities of said active support elements and in said magnitudes of said resistive forces of said active support elements.

47. A system in accordance with claim 46, characterized in that:
said system further comprises means for controlling said changes in said positions to within one micron;
said changes in said positions occur at velocities of up to at least twelve meters/second;
said changes in said velocities occur at accelerations of up to at least 7200 meters/seconds²; and
audible sound produced by said changes in said positions and said velocities is less than one db(A).
